(19) United States
(12) Patent Application Publication
(10) Pub. No.: US 2026/0061066 A1
(43) Pub. Date: Mar. 5, 2026

(54) ANTI-GRP78 ANTIBODY, IMMUNOCONJUGATE AND USES THEREOF

(71) Applicant: UCT BIOSCIENCE CO., LTD., TAIPEI CITY (TW)

(72) Inventors: CHIA-CHENG WU, TAIPEI CITY (TW); YA-WEI TSAI, TAIPEI CITY (TW); CHIA-YING TSAI, TAIPEI CITY (TW); CHIA-HSIANG LO, TAIPEI CITY (TW)

(73) Assignee: UCT BIOSCIENCE CO., LTD., TAIPEI CITY (TW)

(21) Appl. No.: 19/225,449

(22) Filed: Jun. 2, 2025

Related U.S. Application Data
(60) Provisional application No. 63/655,355, filed on Jun. 3, 2024.

(51) Int. Cl. A61K 47/68; A61P 35/00; C07K 16/18; G01N 33/574

(52) U.S. Cl. CPC A61K 47/68037; A61K 47/6843; A61K 47/6889; A61P 35/00; C07K 16/18; G01N 33/5758; C07K 2317/565; G01N 2333/46

(57) ABSTRACT

The present disclosure relates to an anti-glucose-related protein 78 (GRP78) antibody or an antigen-binding fragment thereof. The anti-GRP78 antibody or antigen-binding fragment thereof, such as a single chain variable fragment, can be used as an antigen binding domain of chimeric antigen receptors (CARs) or immunoconjugates for treating and/or preventing a disease and/or disorder caused by or related to GRP78 activity or signaling. The present disclosure also relates to a method or kit for detecting GRP78 or a cancer in a sample.

### PRIORITY INFORMATION

The subject application claims priority to and benefit of U.S. Provisional Patent Application No. 63/655,355, filed Jun. 3, 2024, the content of which is incorporated herein by reference in its entirety.

### REFERENCE TO ELECTRONIC SEQUENCE LISTING

The instant application contains a Sequence Listing which is submitted electronically in .xml format. The Sequence Listing in .xml format was created on May 22, 2025 is named “US15892.xml” and is 14,000 bytes in size. The sequence listing contained in this .xml file is part of the specification and is hereby incorporated herein by reference in its entirety.

### FIELD OF THE INVENTION

The present disclosure relates to an enhanced immunotherapy targeting glucose-related protein 78 (GRP78); more particularly, to an anti-GRP78 antibody, immunoconjugate and uses thereof.

### BACKGROUND OF THE INVENTION

Cancer is a leading cause of death and a significant barrier to increasing life expectancy worldwide. Targeted drug delivery and therapeutic approaches for cancer, aimed at tumors, show promise in reducing or halting tumor progression in living organisms, thereby enabling them to enjoy extended and healthier lifespans. Nevertheless, numerous anti-tumor medications also exhibit toxicity towards non-tumor cells, leading to challenging and undesirable side effects. Consequently, there exists a requirement within the field for a method to selectively deliver anti-tumor agents exclusively to tumor cells, with the objective of minimizing tumor cell proliferation.

Glucose-related protein 78 (GRP78) is primarily a chaperone protein located within the endoplasmic reticulum (ER), assisting in the proper folding of proteins by aiding misfolded protein targeting and facilitating protein assembly. However, under cellular stress, GRP78 is transported to the cell surface (csGRP78), where it interacts with various ligands and initiates diverse intracellular pathways. Consequently, the expression of csGRP78 is associated with cancer and contributes to the maintenance and progression of the disease. Notably, the external exposure of csGRP78 prompts the production of autoantibodies, as observed in patients with prostate or ovarian cancer, indicating that the ability to target csGRP78 has an impact on tumor development. Given that csGRP78 is present on the surface of cancer cells but not normal cells in vivo, it represents a promising target for therapeutic antibody approaches.

However, the production of antibodies specific to GRP78 can be challenging. GRP78 is expressed on the cell surface of embryonic stem cells, and homozygote knockout of GRP78 leads to embryonic lethality, which means that the B cells that produce anti-GRP78 antibodies with high affinity would be subjected to clonal deletion or clonal anergy during normal B cell development. In addition, GRP78 is highly conserved among vertebrate species. Human GRP78 is 99% identical to primate GRP78 and 98% identical to rodent GRP78. This makes it more difficult to generate human anti-GRP78 antibodies with high affinity by animal immunization. Overall, the production of antibodies specific to GRP78 requires careful consideration to ensure the generation of reliable and specific antibodies for research or therapeutic applications.

### SUMMARY OF THE INVENTION

As embodied and broadly described herein, an enhanced immunotherapy for cancer by targeting GRP78 is provided.

The present disclosure relates to an isolated anti-GRP78 antibody or antigen-binding fragment thereof, immunoconjugates and uses thereof.

Accordingly, the present disclosure provides an isolated antibody or antigen-binding fragment thereof that is specific to an epitope in GRP78; wherein the isolated antibody or antigen-binding fragment thereof comprises complementarity determining regions (CDRs) of a heavy chain variable region and/or CDRs of a light chain variable region,
wherein the CDRs of the heavy chain variable region comprise:
CDRH1 having the amino acid sequence of SEQ ID NO: 1 or a substantially similar sequence with at least about 90%, 91%, 92%, 93%, 94%, 95%, 96%, 97%, 98% or 99% identity to SEQ ID NO: 1, CDRH2 having the amino acid sequence of SEQ ID NO: 2 or a substantially similar sequence with at least about 90%, 91%, 92%, 93%, 94%, 95%, 96%, 97%, 98% or 99% identity to SEQ ID NO: 2, and CDRH3 having the amino acid sequence of SEQ ID NO: 3 or a substantially similar sequence with at least about 90%, 91%, 92%, 93%, 94%, 95%, 96%, 97%, 98% or 99% identity to SEQ ID NO: 3; and wherein the CDRs of the light chain variable region comprise:
CDRL1 having the amino acid sequence of SEQ ID NO: 4 or a substantially similar sequence with at least about 90%, 91%, 92%, 93%, 94%, 95%, 96%, 97%, 98% or 99% identity to SEQ ID NO: 4, CDRL2 having the amino acid sequence of SEQ ID NO: 5 or a substantially similar sequence with at least about 90%, 91%, 92%, 93%, 94%, 95%, 96%, 97%, 98% or 99% identity to SEQ ID NO: 5, and CDRL3 having the amino acid sequence of SEQ ID NO: 6 or a substantially similar sequence with at least about 90%, 91%, 92%, 93%, 94%, 95%, 96%, 97%, 98% or 99% identity to SEQ ID NO: 6.

In some embodiments, the heavy chain variable region comprises the amino acid sequence of SEQ ID NO: 7 or a substantially similar sequence having at least about 90%, 91%, 92%, 93%, 94%, 95%, 96%, 97%, 98% or 99% sequence identity; and/or the light chain variable region comprises the amino acid sequence of SEQ ID NO: 8 or a substantially similar sequence having at least about 90%, 91%, 92%, 93%, 94%, 95%, 96%, 97%, 98% or 99% sequence identity.

In some embodiments, the heavy chain comprises the amino acid sequence of SEQ ID NO: 9 or a substantially similar sequence having at least about 90%, 91%, 92%, 93%, 94%, 95%, 96%, 97%, 98% or 99% sequence identity; and/or the light chain comprises the amino acid sequence of SEQ ID NO: 10 or a substantially similar sequence having at least about 90%, 91%, 92%, 93%, 94%, 95%, 96%, 97%, 98% or 99% sequence identity.

In some embodiments of the disclosure, the isolated antibody or antigen-binding fragment thereof is an Fab fragment, an F(ab′) 2 fragment, an scFv fragment, a monoclonal antibody, a chimeric antibody, a nanobody, a composite antibody, a humanized antibody or a human antibody.

In some embodiments of the disclosure, the isolated antibody or antigen-binding fragment is expressed on a surface of a cell. In some embodiments of the disclosure, the cell is an immune cell or a stem cell. In some further embodiments, the cell is a T cell. In another aspect, the stem cell may be an induced pluripotent stem cell.

The disclosure also provides a vector encoding the isolated antibody or antigen-binding fragment thereof.

The disclosure also provides a genetically engineered cell containing the vector as disclosed herein.

The disclosure also provides a genetically engineered cell expressing the isolated antibody or antigen-binding fragment thereof.

In some embodiments of the disclosure, the genetically engineered cell is an immune cell or a stem cell.

In some embodiments, the isolated antibody or antigen-binding fragment thereof is conjugated with a therapeutic agent or a label. In some embodiments, the isolated antibody or antigen-binding fragment thereof is covalently linked to a therapeutic agent or a label.

One aspect of the disclosure relates to an immunoconjugate. An immunoconjugate in accordance with one embodiment of the invention includes:
the isolated antibody or antigen-binding fragment thereof as disclosed herein, and
a therapeutic agent or a label.

In some embodiments of the disclosure, the immunoconjugate has the formula of

Ab-(L-D)ₘ,

wherein Ab represents the isolated antibody or antigen-binding fragment thereof as disclosed herein,
L represents a linker or a direct bond,
D represents the therapeutic agent or the label, and
m represents an integer from 1 to 15, 2 to 14, 3 to 13, 4 to 12, 5 to 11, 6 to 10, 7 to 9, or 8.

Examples of the linker include, but are not limited to, a hydrazone linker, a disulfide bond linker, a maleimidomethyl cyclohexane-1-carboxylate (MCC) linker, a maleimidocaproyl (MC) linker, a Val-Cit (valine-citrulline, VC) linker, a VC-PABC (valine-citrulline-p-aminobenzyl carbamate) linker, a GGFG (Gly-Gly-Phe-Gly) linker, an acid liable carbonate linker, or a sulfo-SPDB (N-succinimidyl 4-(2-pyridyldithio)-2-sulfobutanoate) linker.

Examples of the label include, but are not limited to, a radioisotope, a fluorescence (such as a fluorescence chemical moiety or a fluorescence protein), or a reporter enzyme (such as horseradish peroxidase, HRP).

Examples of the therapeutic agent include, but are not limited to, a calicheamicin, MMAE (monomethyl auristatin E), MMAF (monomethyl auristatin F), emtansine (DM1), ravtansine (DM4), pyrrolobenzodiazepine (PBD) dimer, exatecan, 7-ethyl-10-hydroxycamptothecin (SN38), or deruxtecan (Dxd).

In some embodiments of the disclosure, the therapeutic agent is Dxd and the linker is GGFG.

In some embodiments of the disclosure, the therapeutic agent is Dxd and the linker is VC.

In some embodiments of the disclosure, the therapeutic agent is Dxd and the linker is VC-PABC.

In some embodiments of the disclosure, the linker is conjugated to the isolated antibody or antigen-binding fragment thereof through lysine-conjugation or cysteine-conjugation.

In another aspect, the present disclosure provides a pharmaceutical composition, comprising an effective amount of the isolated antibody or antigen-binding fragment thereof, the vector, the genetically engineered cell or the immunoconjugate as disclosed herein; and optionally a pharmaceutically acceptable carrier.

In another aspect, the present disclosure provides a pharmaceutical composition for use in treating, prophylactically treating and/or preventing a disease and/or disorder caused by or related to GRP78 activity or signaling in a subject in need thereof, comprising an effective amount of the isolated antibody or antigen-binding fragment thereof, the vector, the genetically engineered cell or the immunoconjugate as disclosed herein; and optionally a pharmaceutically acceptable carrier. Alternatively, the present disclosure provides a method for treating, prophylactically treating and/or preventing a disease and/or disorder caused by or related to GRP78 activity or signaling in a subject in need thereof, comprising administering an effective amount of the isolated antibody or antigen-binding fragment thereof, the vector, the genetically engineered cell or the immunoconjugate as disclosed herein; and optionally a pharmaceutically acceptable carrier to the subject.

In some embodiments of the disclosure, the disease is a cancer. Examples of the cancer include, but are not limited to, ovarian cancer, pancreatic cancer, melanoma, myeloma, triple negative breast cancer, T-cell lymphoblastoma, diffuse large B cell lymphoma or acute myeloid leukemia cancer.

In another aspect, the present disclosure provides a method for detecting GRP78 or cancer, comprising contacting a sample with the isolated antibody or antigen-binding fragment thereof or the immunoconjugate as mentioned above.

In another aspect, the present disclosure provides a kit for detecting GRP78 or cancer, comprising the isolated antibody or antigen-binding fragment thereof or the immunoconjugate as mentioned above.

### BRIEF DESCRIPTION OF DRAWINGS

FIG. 1 shows the workflow for obtaining clones of antibodies against GRP78.

FIGS. 2A to 2C show internalization of anti-GRP78 mAbs in different types of cancer cells.

FIG. 3 shows a mass spectrometry profile of G1D10-ADC and DAR analysis.

FIG. 4 shows the binding affinity of two anti-GRP78 antibodies and one ADC, named as G1D10, C38 and G1D10-ADC by ELISA assay.

FIGS. 5A to 5B show in-vivo antitumor activity of G1D10-ADC in a THP-1 xenograft animal model. * indicates a significant (p 0.05) reduction in mean tumor volume compared to vehicle control as determined by two-way ANOVA followed by Bonferroni correction. # indicates a significant anti-tumor activity compared to the vehicle control group as determined by T/C value≤42%.

FIG. 6 shows the level of shed cancer-specific/associated antigen in patients' blood samples.

### DETAILED DESCRIPTION OF THE INVENTION

It is understood that this invention is not limited to the particular materials and methods described herein. It is also to be understood that the terminology used herein is for the purpose of describing particular embodiments and is not intended to limit the scope of the present invention, which will be limited only by the appended claims. Unless otherwise defined, scientific and technical terms used herein shall have the meanings that are commonly understood by those of ordinary skill in the art. Furthermore, unless otherwise required by context, singular terms shall include pluralities and plural terms shall include the singular. Generally, nomenclatures used in connection with, and techniques of, cell and tissue culture, molecular biology, and protein and oligo- or polynucleotide chemistry and hybridization described herein are those well known and commonly used in the art.

The practice of the present disclosure may employ technologies comprising conventional techniques of cell biology, cell culture, antibody technology, and genetic engineering, which are within the ordinary skills of the art. Such techniques are explained fully in the literature.

As utilized in accordance with the present disclosure, the following terms, unless otherwise indicated, shall be understood to have the following meanings: The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually herein.

It must be noted that, as used in this specification and the appended claims, the singular forms “a,” “an,” and “the” include plural referents unless the content clearly dictates otherwise.

The term “antibody,” as used herein, refers to any antigen-binding molecule or molecular complex comprising at least one complementarity determining region (CDR) that is specific to or interacts with a particular antigen (GRP78). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-GRP78 protein antibody (or antigen-binding portion thereof) may be identical to the human germline sequences, or may be naturally or artificially modified. An amino acid consensus sequence may be defined based on a side-by-side analysis of two or more CDRs.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

As used herein, the term “being specific to” or “binding specifically to” means that an antibody does not cross react to a significant extent with other epitopes.

As used herein, the term “epitope” refers to the site on the antigen to which an antibody binds to.

As used herein, the term “complementarity determining region” (CDR) refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity.

As applied to polypeptides, the term “substantial similarity” or “substantially similar” means that two peptide sequences, when optimally aligned, such as by the programs GAP or BESTFIT using default gap weights, share at least 90%, 91%, 92%, 93%, 94%, 95%, 96%, 97%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a non-negative value in the PAM250 log-likelihood matrix.

The term “monoclonal antibody” as used herein is not limited to antibodies produced through hybridoma technology. A monoclonal antibody is derived from a single clone, including any eukaryotic, prokaryotic, or phage clone, by any means available or known in the art.

The term “chimeric” antibody as used herein refers to an antibody having variable sequences derived from a non-human immunoglobulin and human immunoglobulin constant regions, typically chosen from a human immunoglobulin template.

As used herein, the term “an antibody” includes substantially intact antibody molecules, as well as chimeric antibodies, humanized antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include Fv fragments (e.g., single chain Fv and disulphide-bonded Fv) and Fab-like fragments (e.g., Fab fragments, Fab′ fragments and F(ab)₂ fragments). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, scFv and dAb antibody fragments can be expressed in and secreted from E. coli, thus allowing the facile production of large amounts of the said fragments.

“Humanized” forms of non-human antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence.

As used herein, the term “nanobody” refers to an antibody comprising the small single variable domain (VHH) of antibodies obtained from camelids and dromedaries.

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

As used herein, the term “composite antibody” refers to an antibody which has variable regions comprising germline or non-germline immunoglobulin sequences from two or more unrelated variable regions.

As used in the present disclosure, the term “therapeutic agent” refers to any compound, substance, drug or active ingredient having a therapeutic or pharmacological effect that is suitable for administration to a mammal, for example a human.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “T cell” includes CD4+ T cells and CD8+ T cells. The term T cell also includes T helper 1 type T cells, T helper 2 type T cells, T helper 17 type T cells and inhibitory T cells.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). Self-renewal is defined as a stem cell being capable of proliferation and giving rise to more such stem cells, while maintaining its developmental potential. Accordingly, the term “stem cell” refers to any subset of cells that has the developmental potential, under particular circumstances, to differentiate to a more specialized or differentiated phenotype, and which retains the capacity, under certain circumstances, to proliferate without substantially differentiating.

As used herein, the term “immunoconjugate” refers to an antigen-binding protein, e.g., an antibody or antigen-binding fragment, which is chemically or biologically linked to a radioactive agent, a cytokine, an interferon, a target or reporter moiety, an enzyme, a peptide or protein or a therapeutic agent. The antigen-binding protein may be linked to the radioactive agent, cytokine, interferon, target or reporter moiety, enzyme, peptide or therapeutic agent at any location along the molecule so long as it is able to bind its target (GRP78). Examples of immunoconjugates include antibody-drug conjugates and antibody-toxin fusion proteins. In one embodiment of the invention, the agent may be a second, different antibody that binds specifically to GRP78 or an alternative target. The type of therapeutic moiety that may be conjugated to the anti-GRP78 protein (e.g., antibody or fragment) will take into account the condition to be treated and the desired therapeutic effect to be achieved.

As used herein, the term “antibody-drug conjugate” or “ADC” refers to an antigen-binding protein, e.g., an antibody or antigen-binding fragment, which is chemically or biologically linked to a therapeutic agent or the label through a linker. The linker may comprise a cleavable unit or may be non-cleavable. The cleavable unit includes, for example, disulfide containing linkers that are cleavable through disulfide exchange, acid-labile linkers that are cleavable at acidic pH, and linkers that are cleavable by hydrolases (e.g., glycosyl hydrolases such as glucuronidases), esterases, and peptidases (e.g., peptide linkers and glucuronide linkers). The non-cleavable linker is believed to release the therapeutic agent or the label via a proteolytic antibody degradation mechanism.

As used herein, the term “linker” refers to an organic moiety in an immunoconjugate intervening between and covalently attached to a therapeutic agent and an antibody or antigen-binding fragment.

The term “label” means any moiety which can be covalently attached to an antibody and that functions to: (i) provide a detectable signal; (ii) interact with a second label to modify the detectable signal provided by the first or second label, e.g. FRET (fluorescence resonance energy transfer); (iii) stabilize interactions or increase affinity of binding, with antigen or ligand; (iv) affect mobility, e.g. electrophoretic mobility, or cell-permeability, by charge, hydrophobicity, shape, or other physical parameters, or (v) provide a capture moiety, to modulate ligand affinity, antibody/antigen binding, or ionic complexation.

As used herein, the term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “genetically engineered” or “genetic engineering” of cells refers to manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

As used in the present invention, the term “pharmaceutical composition” refers to a mixture containing a therapeutic agent administered to a mammal, for example a human, for preventing, treating, or eliminating a particular disease or pathological condition that the mammal suffers.

As used herein, the term “therapeutically effective amount” or “efficacious amount” refers to the amount of an antibody that, when administered to a mammal or other subject for treating a disease, is sufficient to affect such treatment for the disease.

As used herein, the terms “treatment,” “treating,” and the like, cover any treatment of a disease in a mammal, particularly in a human, and include: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc. Particularly, the subject is vaccinated.

As used herein, the term “in need of treatment” refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a caregiver's expertise, but that includes the knowledge that the subject is ill, or will be ill, as the result of a condition that is treatable by the compounds of the present disclosure.

“Cancer,” “tumor,” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman. Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (7th ed. 2015)). Although transformation can arise from infection with a transforming virus and incorporation of new genomic DNA, or uptake of exogenous DNA, it can also arise spontaneously or following exposure to a carcinogen.

As used herein, the term “sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

The present disclosure provides an antibody that is specific for and has high affinities for an epitope in GRP78, such as human GRP78. The anti-GRP78 antibody or an antigen-binding fragment thereof can deliver therapeutic benefits to a subject. The anti-GRP78 antibody or the antigen-binding fragment thereof according to the disclosure can be used as therapeutics for treating and/or diagnosing a variety of disorders mediated by GRP78, which are more fully described herein.

The antibody or the antigen-binding fragment thereof according to embodiments of the disclosure can be full-length (for example, having a heavy chain constant region selected from the group consisting of IgG1, IgG2 and IgG4 isoforms, and a light chain constant region selected from the group consisting of κ and λ isotypes), or may comprise only an antigen-binding portion (for example, an Fab, F(ab′)₂, or scFv fragment), and may be modified to affect functionalities as needed.

Non-limiting examples of an antigen-binding fragment include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody typically comprises at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

As with a full antibody molecule, an antigen-binding fragment may be monospecific or multi-specific (e.g., bispecific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bispecific antibody formats disclosed herein, may be adapted for use in the context of an antigen-binding fragment of an antibody of the present invention using routine techniques available in the art.

Particularly, the isolated antibody or antigen-binding fragment thereof that is specific for an epitope in GRP78 comprises CDRs of a heavy chain variable region and/or CDRs of a light chain variable region, wherein the CDRs of the heavy chain variable region comprise:
CDRH1 having the amino acid sequence of SEQ ID NO: 1 or the amino acid sequence with at least about 95%, 96%, 97%, 98% or 99% identity to SEQ ID NO: 1, CDRH2 having the amino acid sequence of SEQ ID NO: 2 or the amino acid sequence with at least about 95%, 96%, 97%, 98% or 99% identity to SEQ ID NO: 2, and CDRH3 having the amino acid sequence of SEQ ID NO: 3 or the amino acid sequence with at least about 95%, 96%, 97%, 98% or 99% identity to SEQ ID NO: 3; and
wherein the CDRs of the light chain variable region comprise: CDRL1 having the amino acid sequence of SEQ ID NO: 4 or the amino acid sequence with at least about 95%, 96%, 97%, 98% or 99% identity to SEQ ID NO: 4, CDRL2 having the amino acid sequence of SEQ ID NO: 5 or the amino acid sequence with at least about 95%, 96%, 97%, 98% or 99% identity to SEQ ID NO: 5, and CDRL3 having the amino acid sequence of SEQ ID NO: 6 or the amino acid sequence with at least about 95%, 96%, 97%, 98% or 99% identity to SEQ ID NO: 6.

In some embodiments, the isolated antibody or antigen-binding fragment thereof comprises a heavy chain variable region comprising the amino acid sequence of SEQ ID NO: 7 or a substantially similar sequence thereof; and/or a light chain variable region comprising the amino acid sequence of SEQ ID NO: 8 or a substantially similar sequence thereof. In some further embodiments, the isolated antibody or antigen-binding fragment thereof comprises a heavy chain variable region comprising the amino acid sequence of SEQ ID NO: 7 or a substantially similar sequence thereof having at least 90%, at least 95%, at least 98% or at least 99% sequence identity. In some further embodiments, the isolated antibody or antigen-binding fragment thereof comprises a light chain variable region comprising the amino acid sequence of SEQ ID NO: 8 or a substantially similar sequence thereof having at least 90%, at least 95%, at least 98% or at least 99% sequence identity.

In some embodiments, the heavy chain comprises the amino acid sequence of SEQ ID NO: 9 or a substantially similar sequence having at least about 90%, 91%, 92%, 93%, 94%, 95%, 96%, 97%, 98% or 99% sequence identity; and/or the light chain comprises the amino acid sequence of SEQ ID NO: 10 or a substantially similar sequence having at least about 90%, 91%, 92%, 93%, 94%, 95%, 96%, 97%, 98% or 99% sequence identity.

The sequences in the disclosure are shown in Table 1.

TABLE 1

| | Sequence | SEQ ID NO. |
|---|---|---|
| G1D10_CDRH1 | GFTFSNYAMN | 1 |
| G1D10_CDRH2 | IIYPDSSATYYADSVKG | 2 |
| G1D10_CDRH3 | YHFRWGAMDY | 3 |
| G1D10_CDRL1 | RASQGGSRNLN | 4 |
| G1D10_CDRL2 | KNSNLQS | 5 |
| G1D10_CDRL3 | QQYGSLPWT | 6 |
| G1D10_VH | 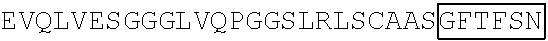 | 7 |
| | 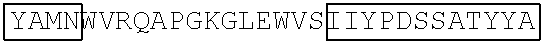 | |
| | 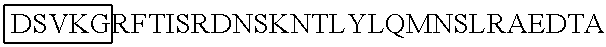 | |
| | 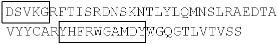 | |
| G1D10_VL | 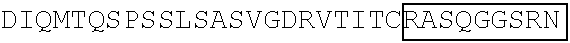 | 8 |
| | 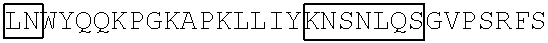 | |
| | 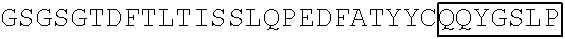 | |
| | 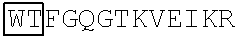 | |
| G1D10_heavy | 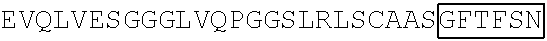 | 9 |
| chain | 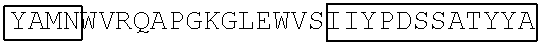 | |
| | 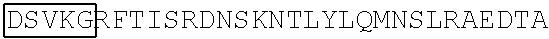 | |
| | 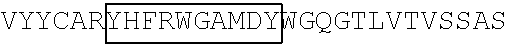 | |
| | TKGPSVFPLAPSSKSTSGGTAALGCLVKDYFP | |
| | EPVTVSWNSGALTSGVHTFPAVLQSSGLYSLS | |
| | SVVTVPSSSLGTQTYICNVNHKPSNTKVDKK | |
| | VEPKSCDKTHTCPPCPAPELLGGPSVFLFPPKP | |
| | KDTLMISRTPEVTCVVVDVSHEDPEVKFNWY | |
| | VDGVEVHNAKTKPREEQYNSTYRVVSVLTV | |
| | LHQDWLNGKEYKCKVSNKALPAPIEKTISKA | |
| | KGQPREPQVYTLPPSREEMTKNQVSLTCLVK | |
| | GFYPSDIAVEWESNGQPENNYKTTPPVLDSD | |
| | GSFFLYSKLTVDKSRWQQGNVFSCSVMHEAL | |
| | HNHYTQKSLSLSPG | |
| G1D10_light | 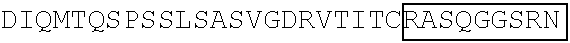 | 10 |
| chain | 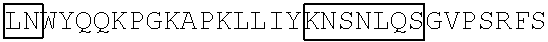 | |
| | 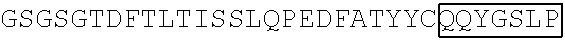 | |
| | 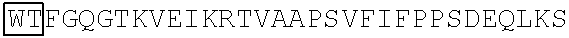 | |
| | GTASVVCLLNNFYPREAKVQWKVDNALQSG | |
| | NSQESVTEQDSKDSTYSLSSTLTLSKADYEKH | |
| | KVYACEVTHQGLSSPVTKSFNRGEC | |
| G1D10 heavy | gaagttcagctggttgaaagcggcggtggtctggttcagcctggtggc | 11 |
| chain | agcctgcgcctgagctgtgcagcaagcggctttaccttttctaactacgc | |
| | aatgaactgggttcgccaggcacctggcaaaggcctggaatgggttag | |
| | catcatttacccagattcttctgcaacctactatgcagatagcgttaaagg | |
| | ccgctttaccattagccgcgataatagcaaaaataccctgtacctgcaaa | |
| | tgaacagcctgcgcgcagaggataccgccgtgtactattgtgcccgct | |
| | accacttccggtggggggctatggattattggggccagggcaccctgg | |
| | tcaccgtgagctctgctagcaccaagggcccttccgtgttccctctggc | |
| | cccttccagcaagtctacctctggcggcaccgctgctctgggctgcctc | |
| | gtgaaggactacttccctgagcctgtgacagtgtcctggaactctggcg | |
| | ccctgacctccggcgtgcacaccttccctgccgtgctgcagtcctccgg | |
| | cctgtactctctgtcctccgtcgtgacagtgccttcctccagcctgggca | |
| | cccagacctacatctgcaacgtgaaccacaagccttccaacaccaagg | |
| | tggacaagaaggtggagcctaagtcctgcgacaagacccacacctgt | |
| | cctccatgccctgcccctgagctgctgggcggaccctccgtgttcctgtt | |
| | ccctccaaagcctaaggacaccctgatgatctcccggacccctgaagt | |
| | gacctgcgtggtggtggacgtgtcccacgaggaccctgaagtgaagtt | |
| | caattggtacgtggacggcgtggaggtgcacaatgctaagaccaagc | |
| | ctcgggaggaacagtacaactccacctaccgggtggtgtccgtgctga | |
| | ccgtgctgcaccaggactggctgaacggcaaagaatacaagtgcaag | |
| | gtgtccaacaaggccctgcctgcccctategaaaagaccatctccaag | |
| | gccaagggccagcctcgggaacctcaggtgtacaccctgcctcccag | |
| | cagggaggagatgaccaagaaccaggtgtccctgacctgtctggttaa | |
| | gggcttctacccttccgacatcgccgtggagtgggagtctaacggcca | |
| | gcctgagaacaactacaagaccacccctcctgtgctggactccgacgg | |
| | ctccttcttcctgtactccaaactgaccgtggacaagtcccggtggcagc | |
| | agggcaacgtgttctcctgctccgtgatgcacgaggccctgcacaacc | |
| | actacacccagaagtccctgtctctgtctcctggctga | |
| G1D10 light | gatatccagatgacacagagccctagcagcctgtctgccagcgtgggc | 12 |
| chain | gatcgcgtgaccatcacctgtagagccagccagggtggttctcgcaac | |
| | ctgaactggtatcagcagaagcctggcaaagcccctaaactgctgatct | |
| | ataaaaactctaacctgcagtctggcgtgccaagccgcttttctggcagc | |
| | ggctctggcaccgacttcaccctgaccatctctagcctgcagcctgaag | |
| | acttcgccacctattattgccagcagtacggttctttgccatggacctttg | |
| | gccagggcaccaaagtggaaatcaaacgcacggtggctgcaccatct | |
| | gtcttcatcttcccgccatctgatgagcagttgaaatctggaactgcctct | |
| | gttgtgtgcctgctgaataacttctatcccagagaggccaaagtacagtg | |
| | gaaggtggataacgccctccaatcgggtaactcccaggagagtgtcac | |
| | agagcaggacagcaaggacagcacctacagcctcagcagcaccctg | |
| | acgctgagcaaagcagactacgagaaacacaaagtctacgcctgcga | |
| | agtcacccatcagggcctgagctcgcccgtcacaaagagcttcaacag | |
| | gggagagtgttga | |

In some embodiments of the present disclosure, the isolated antibody or antigen-binding fragment thereof includes the heavy chain variable region comprising the amino acid sequence of SEQ ID NO: 7, and the light chain variable region comprising the amino acid sequence of SEQ ID NO: 8.

In some embodiments of the present disclosure, the isolated antibody or antigen-binding fragment thereof includes the heavy chain comprising the amino acid sequence of SEQ ID NO: 9, and the light chain comprising the amino acid sequence of SEQ ID NO: 10.

The isolated antibody or antigen-binding fragment thereof disclosed herein may comprise one or more amino acid substitutions, insertions and/or deletions in the frameworks and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes an antibody, and an antigen-binding fragment thereof, which are derived from any of the amino acid sequences disclosed herein, wherein one or more amino acids within one or more frameworks and/or CDR regions are mutated to the corresponding residue(s) of the germline sequence from which the antibody was derived, or to the corresponding residue(s) of another mammalian germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the frameworks and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the frameworks and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the frameworks and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired properties, such as improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The isolated antibody or antigen-binding fragment thereof of the present disclosure may be monospecific, bi-specific, or multispecific. Multispecific antibodies may be specific for different epitopes of one target polypeptide or may contain antigen-binding domains specific for more than one target polypeptide. The anti-GRP78 antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present disclosure includes bi-specific antibodies wherein one arm of an immunoglobulin is specific for GRP78 or a fragment thereof, and the other arm of the immunoglobulin is specific for a second target or is conjugated to a therapeutic agent.

The genes encoding the heavy and light chains of an antibody of interest can be cloned from a cell, e.g., the genes encoding a monoclonal antibody can be cloned and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)).

In some embodiments of the disclosure, the heavy chain is coded by a polynucleotide of SEQ ID NO: 11 and/or the light chain is coded by a polynucleotide of SEQ ID NO: 12.

An example of a method for manufacturing the antibody or antigen-binding fragment comprises: (a) introducing into a host cell one or more polynucleotides encoding said isolated antibody or antigen-binding fragment; (b) culturing the host cell under conditions favorable to expression of the one or more polynucleotides; and (c) optionally, isolating the antibody or antigen-binding fragment from the host cell and/or a medium in which the host cell is grown.

The isolated antibody or antigen-binding fragment thereof may be encoded in a vector. The present disclosure also provides a vector encoding the anti-GRP78 antibody or the antigen-binding fragment thereof as described herein. In one embodiment, one type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In another aspect, the present disclosure provides a genetically engineered cell expressing the isolated antibody or antigen-binding fragment thereof as described herein or containing the vector as described herein. The genetically engineered cell may be an immune cell, such as a T cell, NK cell, macrophage, or a stem cell. In one embodiment of the disclosure, the isolated antibody or antigen-binding fragment thereof is expressed on the surface of a cell. Particularly, the cell is a T-cell, NK cell, macrophage, or a stem cell.

In some embodiments of the disclosure, the isolated antibody or antigen-binding fragment thereof is in the form of a chimeric antigen receptor.

The term “chimeric antigen receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains.

In some embodiments of the disclosure, the genetically engineered cell comprises the isolated antibody or antigen-binding fragment thereof as described herein, a transmembrane domain, and a CD3 zeta signaling domain. In some further embodiments of the disclosure, wherein the isolated antibody or antigen-binding fragment thereof is selected from the group consisting of a human antibody, a humanized antibody, an antigen binding fragment thereof, and any combination thereof.

In some embodiments of the disclosure, the genetically engineered cell further comprises a costimulatory signaling region comprising the intracellular domain of a costimulatory molecule selected from the group consisting of CD27, CD28, 4-1BB, OX40, CD30, CD40, PD-1, lymphocyte function-associated antigen-1 (LFA-1), CD2, CD7, LIGHT, NKG2C, B7-H3, a ligand that specifically binds with CD83, and any combination thereof.

One aspect of the disclosure relates to an immunoconjugate. An immunoconjugate in accordance with one embodiment of the disclosure includes: the isolated antibody or antigen-binding fragment thereof as disclosed herein, and a therapeutic agent or a label.

In some embodiments of the disclosure, the immunoconjugate has the formula of

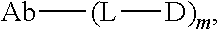

wherein Ab represents the isolated antibody or antigen-binding fragment thereof as disclosed herein,
L represents a linker or a direct bond,
D represents the therapeutic agent or the label, and
m represents an integer from 1 to 15, 2 to 14, 3 to 13, 4 to 12, 5 to 11, 6 to 10, 7 to 9, or 8.

Embodiments of the disclosure relate to antibody-drug conjugates containing GRP78 antibodies and their uses in therapy. The expression of cell surface GRP78 is associated with cancer and contributes to the maintenance and progression of the disease. Therefore, ADCs based on antibodies against GRP78 can be useful diagnostic and/or treatment agents.

However, the fast internalization or lack of ADCC activity of a therapeutic antibody might result in the antibody being ineffective and lead to resistance. Therefore, there is a need to enhance the therapeutic efficacy of anti-GRP78 based therapeutics. One approach is to conjugate a payload with an anti-GRP78 antibody (i.e., an antibody-drug conjugate). By conjugating anti-GRP78 antibodies to payloads (i.e., ADCs), embodiments of the disclosure are more potent than the naked anti-GRP78 antibodies, thereby enabling the use of fewer antibodies.

In some embodiments of the present disclosure, the isolated anti-GRP78 antibody or the antigen-binding fragment thereof may be used as antibody-drug conjugates (ADCs), which can specifically target GRP78. That is, the present disclosure also provides an immunoconjugate, including the aforementioned isolated anti-GRP78 antibody or the antigen-binding fragment thereof, and the therapeutic agent conjugated with the isolated anti-GRP78 antibody or the antigen-binding fragment thereof. The therapeutic agent or payload can be any that are commonly used in ADCs.

In some embodiments of the disclosure, the therapeutic agent represents a cytostatic or cytotoxic agent or an isotope-chelating agent with corresponding radioisotopes. Examples of the cytostatic or cytotoxic agent include, without limitation, antimetabolites (e.g., fluorouracil (5-FU), floxuridine (5-FUdR), methotrexate, leucovorin, hydroxyurca, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed); alkylating agents (e.g., cmelphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, carboplatin, nedaplatin, oxaliplatin, satraplatin, or triplatin); DNA minor groove alkylating agents (e.g., duocarmycins such as CC-1065, and any analogs or derivatives thereof; pyrrolobenzodiazapenes, or any analogs or derivatives thereof); anthracyclines (e.g., daunorubicin, doxorubicin, epirubicin, idarubicin, or valrubicin); antibiotics (e.g., dactinomycin, bleomycin, mithramycin, anthramycin, streptozotocin, gramicidin D, mitomycins (e.g., mitomycin C); calicheamicins; antimitotic agents (including, e.g., maytansinoids (such as DM1, DM3, and DM4), auristatins (including, e.g., monomethyl auristatin E (MMAE) and monomethyl auristatin F (MMAF)), dolastatins, cryptophycins, vinca alkaloids (e.g., vincristine, vinblastine, vindesine, vinorelbine), taxanes (e.g., paclitaxel, docetaxel, or a novel taxane), tubulysins, and colchicines); topoisomerase inhibitors (e.g., irinotecan, topotecan, SN38, exatecan, deruxtecan, Dxd, camptothecin, etoposide, teniposide, amsacrine, or mitoxantrone); HDAC inhibitor (e.g., vorinostat, romidepsin, chidamide, panobinostat, or belinostat); proteasome inhibitors (e.g., peptidyl boronic acids); as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Examples of the isotope-chelating agents include, without limitation, ethylenediaminetetraacetic acid (EDTA), diethylenetriamine-N,N,N′,N″,N″-pentaacetate (DTPA), 1,4,7,10-tetraazacyclododecane-N,N′,N″,N′″-tetraacetate (DOTA), 1,4,7,10-tetrakis(2-hydroxypropyl)-1,4,7,10-tetraazacyclododecane (THP), triethylenetetraamine-N,N,N′,N″,N″,N′″-hexaacetate (TTHA), 1,4,7,10-tetraazacyclododecane-N,N′,N″,N′″-tetrakis(methylenephosphonate) (DOTP), and mercaptoacetyltriglycine (MAG3).

In some embodiments of the disclosure, the linker has a functionality that is capable of reacting with a free cysteine or ε-NH₂ of lysine present in the isolated antibody or antigen-binding fragment thereof to form a covalent bond as cysteine-conjugation or lysine-conjugation. Non-limiting examples of such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, for example, the conjugation method on page 766 of Klussman, et al. (2004), Bioconjugate Chemistry 15(4):765-773, and the Examples herein.

Suitable labels include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In some embodiments, the linker has a functionality that is capable of reacting with an electrophilic group present on the isolated antibody or antigen-binding fragment thereof. Examples of such electrophilic groups include, but are not limited to, aldehyde and ketone carbonyl groups. In some embodiments, a heteroatom of the reactive functionality of the linker is capable of reacting with an electrophilic group on the isolated antibody or antigen-binding fragment thereof and forming a covalent bond to the isolated antibody or antigen-binding fragment thereof unit. Non-limiting examples of such reactive functionalities include, but are not limited to, hydrazide, oxime, amino, hydrazine, thiosemicarbazone, hydrazine carboxylate, and arylhydrazide.

The linker may comprise one or more linker moieties. Exemplary linker moieties include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-succinimidyl 4-(2-pyridylthio) pentanoate (“SPP”), and 4-(N-maleimidomethyl)cyclohexane-1-carboxylate (“MCC”). Various linker moieties are known in the art, some of which are described below.

The linker may be a “cleavable linker,” facilitating release of a therapeutic agent or a label. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al, Cancer Research 52:127-131 (1992); U.S. Pat. No. 5,208,020).

In some embodiments, the linker comprises an “amino acid unit.” In some such embodiments, the amino acid unit allows for cleavage of the linker by a protease, thereby facilitating release of the therapeutic agent or label from the immunoconjugate upon exposure to intracellular proteases, such as lysosomal enzymes (Doronina et al, (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some aspects, the linker comprises a self-immolative linker. In some aspects, the self-immolative linker of the present disclosure undergoes 1,4 elimination after the enzymatic cleavage of the protease-cleavable linker. In some aspects, the self-immolative linker of the present disclosure undergoes 1,6 elimination after the enzymatic cleavage of the protease-cleavable linker. In some aspects, the self-immolative linker is, e.g., a p-aminobenzyl (pAB) derivative, such as a p-aminobenzyl carbamate (pABC), a p-amino benzyl ether (PABE), a p-amino benzyl carbonate, or a combination thereof. In certain aspects, the self-immolative linker comprises an aromatic group. In some aspects, the aromatic group is selected from the group consisting of benzyl, cinnamyl, naphthyl, and biphenyl. In some aspects, the aromatic group is heterocyclic. In other aspects, the aromatic group comprises at least one substituent. In some aspects, the at least one substituent is selected from the group consisting of F, Cl, I, Br, OH, methyl, methoxy, NO₂, NH₂, NO₃⁺, NHCOCH₃, N(CH₃)₂, NHCOCF₃, alkyl, haloalkyl, C1-C8 alkylhalide, carboxylate, sulfate, sulfamate, and sulfonate. In other aspects, at least one C in the aromatic group is substituted with N, O, or C—R*, wherein R* is independently selected from H, F, Cl, I, Br, OH, methyl, methoxy, NO₂, NH₂, NO₃⁺, NHCOCH₃, N(CH₃)₂, NHCOCF₃, alkyl, haloalkyl, C1-C8 alkylhalide, carboxylate, sulfate, sulfamate, and sulfonate.

In some embodiments, the linker is selected from the group consisting: N-succinimidyl 4-(2-pyridyldithio) pentanoate (SPP) or N-succinimidyl 4-(2-pyridyldithio)-2-sulfopentanoate (sulfo-SPP); N-succinimidyl 4-(2-pyridyldithio) butanoate (SPDB) or N-succinimidyl 4-(2-pyridyldithio)-2-sulfobutanoate (sulfo-SPDB); N-succinimidyl 4-(malcimidomethyl)cyclohexanecarboxylate (SMCC); N-sulfosuccinimidyl 4-(maleimidomethyl)cyclohexanecarboxylate (sulfo SMCC); N-succinimidyl-4-(iodoacetyl)-aminobenzoate (SIAB); and N-succinimidyl-[(N-malcimidopropionamido)-tetracthyleneglycol] ester (NHS-PEG4-maleimide). In another embodiment, the linker is N-succinimidyl 4-(2-pyridyldithio)-2-sulfobutanoate (sulfo-SPDB).

The disclosure further provides pharmaceutical compositions including the isolated antibody or antigen-binding fragment thereof, and the genetically engineered cell or the immunoconjugate as described herein. In some embodiments of the disclosure, the pharmaceutical compositions as described herein are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. When an antibody of the present disclosure is used for treating a condition or disease associated with GRP78 in an adult patient, it may be advantageous to intravenously administer the antibody of the present disclosure. Depending on the severity of the condition, the frequency and the duration of the treatment can be adjusted. Effective dosages and schedules for administering the antibody may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once the entire pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1984, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. Examples of suitable aqueous injection mediums include physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. Examples of suitable oily mediums include sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

In some embodiments of the disclosure, the pharmaceutical composition is for use in treating, prophylactically treating and/or preventing a disease and/or disorder caused by or related to GRP78 activity and/or signaling. Alternatively, the present disclosure also provides a method for treating, prophylactically treating and/or preventing a disease and/or disorder caused by or related to GRP78 activity and/or signaling in a subject in need of such treatment, comprising administering to the subject the pharmaceutical composition. In some embodiments, the disease is a cancer, such as solid cancer, including lung cancer, breast cancer, prostate cancer, colorectal cancer, gastric cancer, pancreatic cancer, ovarian cancer, hepatocellular carcinoma, renal cell carcinoma, testicular cancer, melanoma, and papillary thyroid cancer, and non-solid cancer such as leukemia, myeloma, and lymphoma.

The present disclosure further provides a method for detecting GRP78 or cancer in a sample, which includes contacting a sample with the isolated antibody or antigen-binding fragment thereof or the immunoconjugate as described herein. The present disclosure also provides a kit for detecting GRP78 in a sample, including the isolated antibody or antigen-binding fragment thereof or the immunoconjugate as described herein.

The isolated antibody or antigen-binding fragment thereof as described herein may also be used to detect and/or measure GRP78, or GRP78-expressing cells in a sample, e.g., for diagnostic purposes. For example, an anti-GRP78 antibody, or the antigen-binding fragment thereof, may be used to diagnose a condition or disease characterized by aberrant expression (e.g., over-expression, under-expression, lack of expression, etc.) of GRP78. Exemplary diagnostic assays for GRP78 may comprise, e.g., contacting a sample, obtained from a patient, with an anti-GRP78 antibody of the disclosure, wherein the anti-GRP78 antibody is labeled with a detectable label or reporter molecule. Alternatively, an unlabeled anti-GRP78 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure GRP78 in a sample include enzyme-linked immunosorbent assay (ELISA), electrochemiluminescence immunoassay (ECLIA), radioimmunoassay (RIA), and fluorescence-activated cell sorting (FACS).

The following examples are provided to aid those skilled in the art in practicing the present disclosure.

### EXAMPLES

The following examples illustrate the development and use of GRP78-specific antibodies to suppress tumor growth by inducing an anti-GRP78 immune response.

### Example 1 Generation of Anti-GRP78 Monoclonal Antibody (G1D10)

1-1 Preparation of Fully Synthetic Human scFv Phage for Bio-Panning

The workflow for obtaining clones of antibodies against GRP78 may be as shown in FIG. 1. The fully synthetic human scFv phage library was inoculated with bacteria into a 2×YT medium containing 100 μg/ml ampicillin and 2% glucose (2YTAG) and incubated by shaking at 37° C. until the OD at 600 nm reaches 0.5. The culture was infected with a helper phage and then cultured without shaking in a 37° C. water bath for 30 min. The cells in the culture were collected and suspended in a 2×YT medium containing 100 μg/ml ampicillin and 25 μg/ml kanamycin (2YTAK) and further incubated with shaking at 30° C. overnight. The supernatant of the culture was collected and mixed with 1/5 volume of PEG/NaCl (20% Polyethylene glycol 8000, 2.5 M NaCl) and stayed at 4° C. for at least one hour. After centrifuging, the pellet was collected and suspended in PBS and spun again to collect the supernatant.

1-2 Bio-Panning of scFv Phages by Using ELISA Method

An ELISA plate (Nunc) was coated with 5 to 25 μg/100 μl of antigen (HSPA5/GRP78 (ACTIVE PROTEIN™) Human Protein (ORIGENE™, Cat. No. AR03021PU-L) per well and stayed in sodium bicarbonate buffer (pH 9.6) at 4° C. overnight. The wells were washed 3 times with PBS and blocked with 300 μl of 5% skim milk-containing PBS (MPBS) per well at 37° C. for 2 hours. After being washed 3 times with PBS, 100 μl of phages in 5% MPBS with His-tag-containing fusion protein were added and incubated at 37° C. for 90 min. After being washed 4 to 10 times with 0.05% Tween 20-containing PBS (PBST) and 4 to 10 times with PBS, the phages were eluted by adding 100 μl of 100 mM triethylamine (TEA) and reacted at 37° C. for 20 min. 100 μl of eluted phages were neutralized with 50 μl of 1 M Tris, pH 7.4. 3 mL of TG1 at an exponentially growing stage were added with the eluted phages. The cultures were incubated at 37° C. for 30 min without shaking for infection. The infected TG1 bacteria were added with 20 mL of 2×YT-AG and then incubated at 37° C. overnight.

### 1-3 Preparation of Next Round Phage

The cultures provided by Example 1-2 were spun, collected, and then suspended in 0.5 mL of 2×YT-AG, 15% glycerol. Then, 10 μl of the bacteria were added to 10 mL of 2×YT-AG and the bacteria grew with shaking at 37° C. until the OD at 600 nm reaches 0.5. 10 mL of the culture was infected with M13KO7 helper phage by adding the helper phage at a ratio of 1:20 (M13KO7 helper phage: culture) and the infected culture was incubated without shaking in a 37° C. water bath for 30 min. The cultures were spun to collect the pellet, and the pellet was suspended with 25 mL of 2×YT-AK and then cultured at 30° C. overnight. Further, 25 mL of the overnight culture was spun at 10,000 rpm for 20 min to collect the supernatant, and 1/5 volume (5 mL) PEG/NaCl was added to the supernatant to provide a mixture. The mixture was spun at 10,000 rpm for 20 min and the pellet was collected and suspended in 0.5 mL PBS.

1-4 Screening of Human GRP78 Specific scFv-Phage Clones by ELISA

The suspensions provided by Example 1-3 were spread on the plate, and cultured to obtain individual colonies. The individual colonies thus provided were inoculated into 200 μl of 2×YT-AG 96-well plates and grew with shaking at 37° C. overnight, and then 10 μl of the culture was transferred to a second 96-well plate containing 180 μl of 2×YT-A per well for shaking at 37° C. for 2 hours. Then, 50 μl of 2×YT-A with 2.4×10¹⁰ pfu/mL M13KO7 helper phage was added to each well of the second plate to provide a mixture. The mixture was shaken at 37° C. for 2 hours. 50 μl of 2×YT-AK3 (the kanamycin concentration was 300 μg/mL) was added to the mixture, and then grew with shaking at 30° C. overnight. The culture was added with 50 μl MPBS to provide a phage mixture, and 100 μl of the phage mixture was taken for phage ELISA.

The ELISA plates were coated with 1 μg/mL of protein antigen per well, and then rinsed 3 times with PBS and blocked with 300 μl of 5% MPBS per well at 37° C. for 2 hours. After rinsing a further 3 times with PBS, 100 μl phage mixture as detailed above was added and incubated at 37° C. for 90 min. The phage solution was discarded and the wells were washed 6 times with PBST and 6 times with PBS, and then, an appropriate diluted HRP-anti-M13 antibody in 5% MPBS was added to provide a mixture. The mixture was incubated at 37° C. for 60 min, and then washed 6 times with PBST. The wells were developed with substrate solution (TMB) and the reactions were stopped by adding 100 μl of 1 M Hydrochloric acid. The color turned yellow, and the OD at 650 nm and at 450 nm was assayed.

### 1-5 The Monoclonal Phage Preparation

The clones provided by Example 1-4 were subjected to the following procedure. The bacteria were cultured at 37° C. overnight. Thereafter, 100 μl bacteria were added with 2×YT-AG, and the bacteria grew with shaking at 37° C. until the OD at 600 nm reaches 0.5. 10 mL of the culture was infected with M13KO7 helper phage by adding the helper phage at a ratio of 1:20 (M13KO7 helper phage: culture), and the infected culture was incubated without shaking in a 37° C. water bath for 30 min. The cultures were spun to collect the pellet, and the pellet was suspended with 25 mL of 2×YT-AK and then cultured at 30° C. overnight. Further, 25 mL of the overnight culture was spun at 10,000 rpm for 20 min to collect the supernatant, and 1/5 volume (5 ml) PEG/NaCl was added to the supernatant to provide a mixture. The mixture was spun at 10,000 rpm for 20 min, and the pellet was collected and suspended in 0.5 mL PBS.

1-6 Cloning of Anti-GRP78 Antibody Genes into IgG1 Expression Vector

The insert DNA PCR with specific primers designed for VH and VL on phagemid was prepared. The IgG1 expression vector with restriction enzymes DraIII-HF and BsiWI-HF was digested, and then the vector DNA was purified using a gel extraction kit. Next, the vector DNA by NheI-HF and MluI-HF was digested. The insert and vector by T4 DNA polymerase was ligated, and the ligated plasmid was transformed into DH5a host cells for amplification and isolation.

### 1-7 Expression of Full-Length Antibodies

The genes encoding anti-human GRP78 antibodies were constructed by inserting the VH and VL chains, which come from the scFv phage clones provided by Example 1-5, into an expression vector containing CH and CL chain, respectively. Frec-style 293 cells were transfected with the constructed vector. The antibodies were purified by using Protein A Sepharose Fast Flow (GE™ Healthcare, 17-1279-02). After purification, the antibodies were quantified by measuring at OD 280 nm and checked by reducing and non-reducing PAGE. The anti-GRP78 antibody was named as G1D10, and the sequences of CDRs, the heavy chain variable region and light chain variable region are shown in Table 1.

### Example 2 Anti-GRP78 Antibody G1D10 Binds to Different Cancer Cell Lines, as Demonstrated by Flow Cytometry

Ovarian cancer cells were analyzed by flow cytometry for cell surface GRP78 expression. The tumor cells were assessed at 4° C. 1 hr using GRP78 specific antibody, followed by Alexa Fluor 488 conjugated secondary antibodies (JACKSON IMMUNO™, 109-545-003, 115-545-003) and analysis by flow cytometry (CYTOFLEX™, BECKMAN). The anti-GRP78 antibodies, C38 (eBioscience, Cat. No. 14-9768-82) and G1D10 displayed binding to the cell surface of OVCAR3 cell line. The anti-GRP78 antibodies G1D10 (30 μg/mL) bound to the cell surfaces of OVCAR3 at a higher level than the conventional anti-GRP78 antibody C38 (30 μg/mL), as shown in Table 2.

TABLE 2

| | | | C38 | |
|---|---|---|---|---|
| | | A488-anti- | (30 | |
| Cancer type | Cell line | mouse-IgG | mg/ml) | |
| OVARIAN CANCER | OVCAR3 | 1.8% | 2.3% | |
| | | | | G2D03 |
| | | A488-anti- | | (30 |
| | Cell line | human-IgG | | mg/ml) |
| | OVCAR3 | 1.2% | | 23.7% |
| | OV90 | 0.8% | | 19.6% |
| | SKOV3MT1 | 0.9% | | 59.1% |
| PANCREATIC | PL45 | 1.1% | | 21.3% |
| CANCER | AsPC-1 | 0.9% | | 22.3% |
| | KLM-1 | 1.0% | | 43.9% |
| TRIPLE NEGATIVE | MDA-MB- | 1.2% | | 27.3% |
| BREST CANCER | 231 | | | |
| (TNBC) | HCC1806 | 1.2% | | 50.3% |
| T-CELL ACUTE | CEM | 0.5% | | 23.6% |
| LYMPHOBLAST | | | | |
| LEUKEMIA | | | | |
| (T-ALL) | | | | |
| DIFFUSE LARGE B | Raji | 2.2% | | 24.5% |
| CELL LYMPHOMA | | | | |
| (DLBCL) | | | | |
| MELANOMA | G361 | 0.7% | | 63.8% |
| MULTIPLE | RPMI8226 | 0.7% | | 74% |
| MYELOMA (MM) | NCI-H929 | 1.4% | | 46.5% |
| | MM.1S | 1.0% | | 40.5% |
| ACUTE MYELOID | THP-1 | 1.0% | | 100% |
| LEUKEMIA | HL-60 | 1.0% | | 98.4% |
| (AML) | | | | |

Different types of cancer cells were analyzed by flow cytometry for cell surface GRP78 expression. The anti-GRP78 antibody G1D10 (30 μg/mL) displayed binding to the cell surface of ovarian cancer cells (OV-90, SKOV3MT1), pancreatic cancer cells (PL45, AsPC-1, KLM-1), triple negative breast cancer cells (MDA-MB-231, HCC1806), T lymphoblast cell line (CEM), Diffuse large B cell lymphoma (DLBCL) cell line (Raji), melanoma cells (G-361), myeloma cells (RPMI 8226, NCI-H929, MM.1S), and acute myeloid leukemia cancer cells (THP-1, HL-60), as shown in Table 2.

### Example 3 Internalization of Anti-GRP78 mAbs in Different Types of Cancer Cells

Receptor-mediated internalization of antibodies can provide cell-specific drug delivery. The internalization is necessary for some targeted therapies using ADC. It is known in the art that internalization of ADCs is both antibody-dependent and payload-dependent. That is, not all antibodies can provide a delivery mechanism for ADCs. Similarly, different payloads on the same antibody may have dramatically different internalization efficiencies.

Internalization and degradation of anti-GRP78 ADC and anti-GRP78 antibody can be measured by flow cytometry. Two methods were used in this example: directly fluorescent-labeling on antibodies or the use of fluorescent-conjugated secondary antibodies to detect the primary antibodies left on the cell surface after internalization.

Briefly, cancer cells were seeded in a 1×10⁵ cell/well. Next, 0.5-1 mg of fluorescent-labelled anti-GRP78 antibody was added to 100 μl of target cell (cell density 1×10⁶ cells/ml) in FACS buffer for 1 hour at 4° C. to enable specific binding of anti-GRP78 antibody to the cell surface targets. After incubation, the cells were washed three times with FACS buffer to remove unbound antibody. The cells were then incubated at 37° C. with 5% CO₂ for antibody internalization.

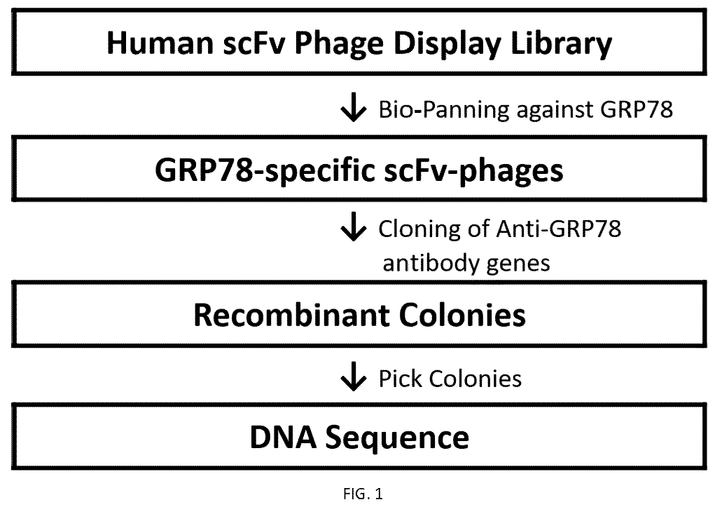

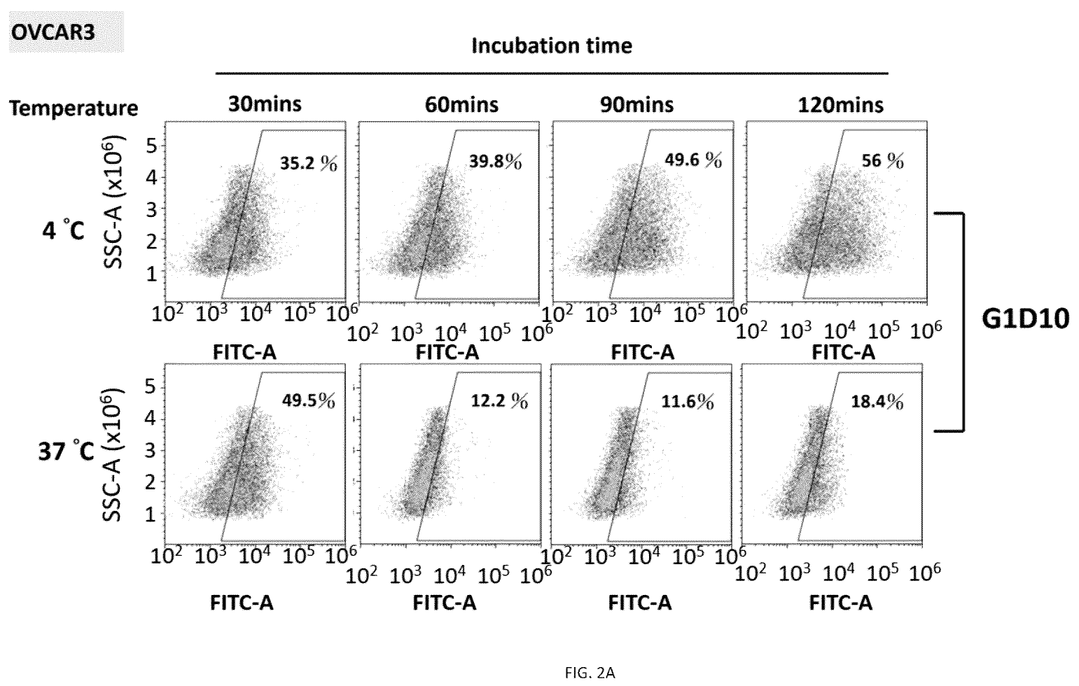

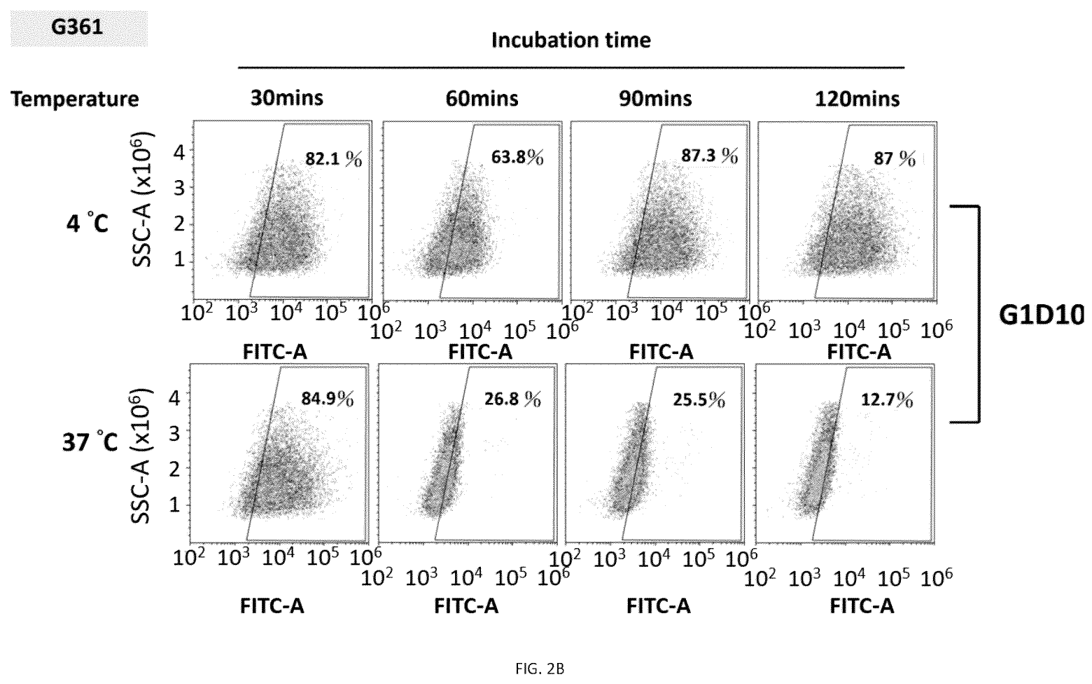

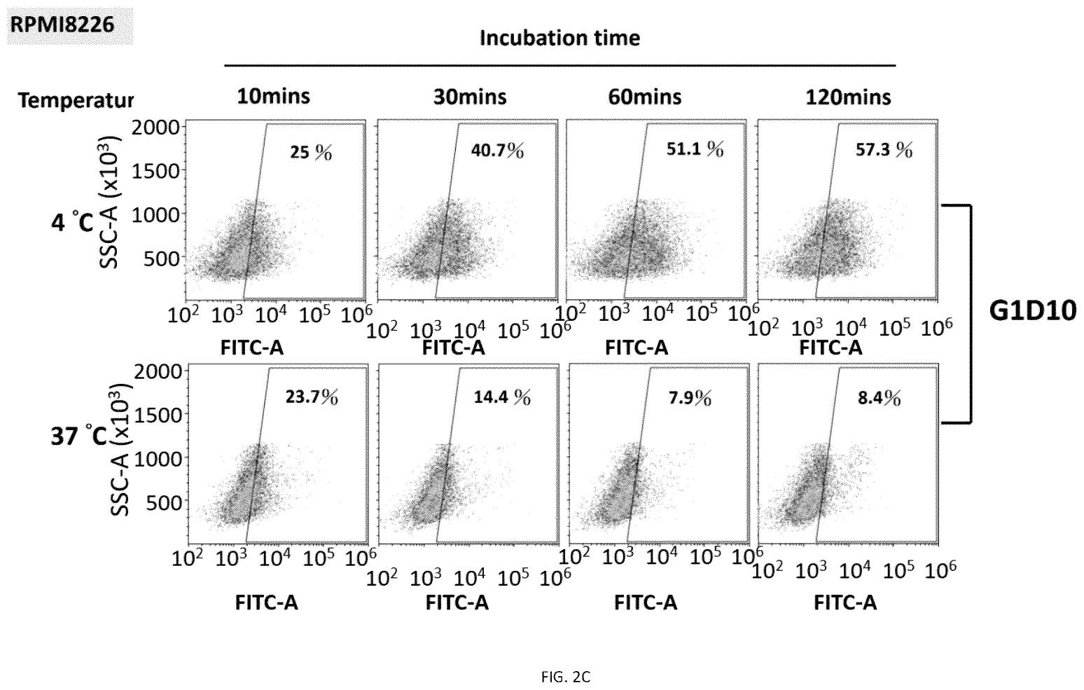

Due to poor binding ability of C38 (EBIOSCIENCE™, Cat. No. 14-9768-82), it is hard to demonstrate its internalization. However, in some embodiments of this invention, G1D10 was internalized into Ovarian cancer cells (OVCAR3) (FIG. 2A), melanoma cells (G-361) (FIG. 2B), and myeloma cells (RPMI 8226) (FIG. 2C) during 37° C. incubation confirmed by flow cytometry. G1D10 (30 μg/mL) incubated at 37° C. to allow the antibodies to internalize, while incubated at 4° C. to prevent the internalization of the antibody. The samples were collected at various time points (10, 30, 60, 90 and 120 mins) and determined by comparing the decrease cell surface-bound antibody of samples incubated at 37° C. to control samples incubated at 4° C.

### Example 4 Preparation of Anti-GRP78 Antibody-SMCC-DM1 Conjugates

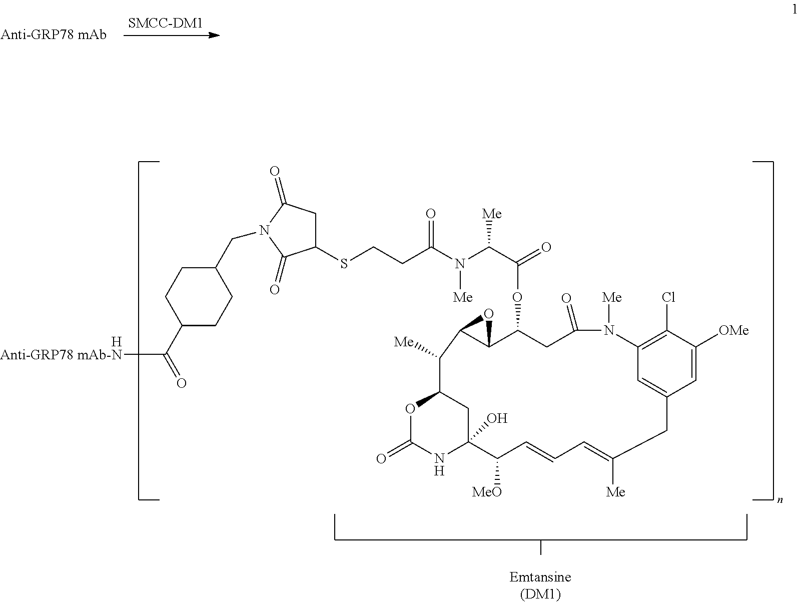

ADCs can be produced by traditional stochastic coupling on exposed lysine or reduced interchain cysteine residues or via site-specific conjugation approaches, like glycoconjugation. The latter yields more homogeneous ADCs in terms of DAR and conjugation site than randomly linked ADCs.

In this example, ADC contains DM1, which is a maytansinoid that was developed for cancer therapy. Maytansine, a benzoansamacrolide, is a highly potent microtubule-targeted compound that induces mitotic arrest and kills tumor cells at subnanomolar concentrations. DM1 binds at the tips of microtubules to suppress the dynamicity of microtubules, i.e., inhibiting the assembly of microtubules. DM1 is a maytansinoid with less systemic toxicity than maytansine. In this example, SMCC-DM1, which is DM1 with a reactive linker SMCC, is used to react with antibody to make antibody drug conjugates. SMCC-DM1 is available from commercial sources, such as MEDKOO BIOSCIENCES™, Inc. or ALB Technology™.

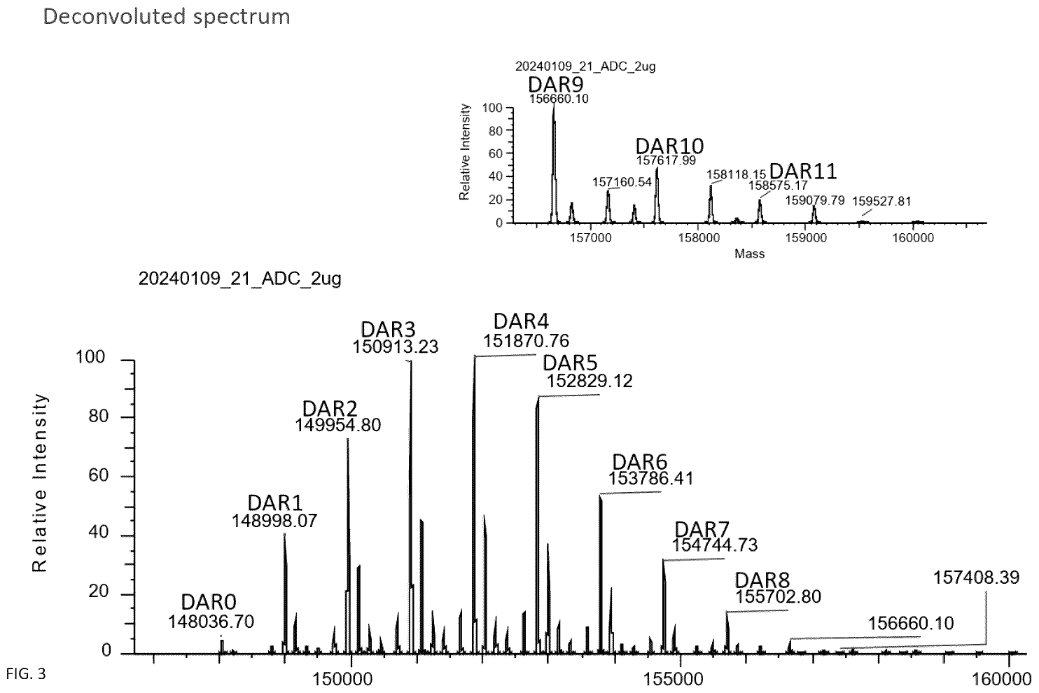

For example, the pH 6.5 conjugation buffer containing 25 mM sodium citrate is first prepared to make an appropriate environment for the synthesis. G1D10 antibody at 66.67 nmol (4.5 mg/mL, 2.23 mL) was mixed with 12 mol eq (800 nmol, 0.16 mL) of 5 mM SMCC-DM1 in dimethylacetamide (DMA). Additional DMA is added to reach a 16.67% organic solution. Following a 1.5 hours incubation at 37° C., the solution is then purified using desalting SEPHADEX™ G-25 column in Storage buffer. Lysine-based G1D10-ADC conjugation produces a heterogeneous population with a range of drug-to-antibody (DAR) ratios. We use intact LC/MS to analyze the drug-loading profile of each ADC G1D10-DM1 and quantify the average DAR as shown in the deconvoluted mass spectrum (FIG. 3).

### Example 5 Preparation of Anti-GRP78 Antibody-VC-PABC-MMAE Conjugates

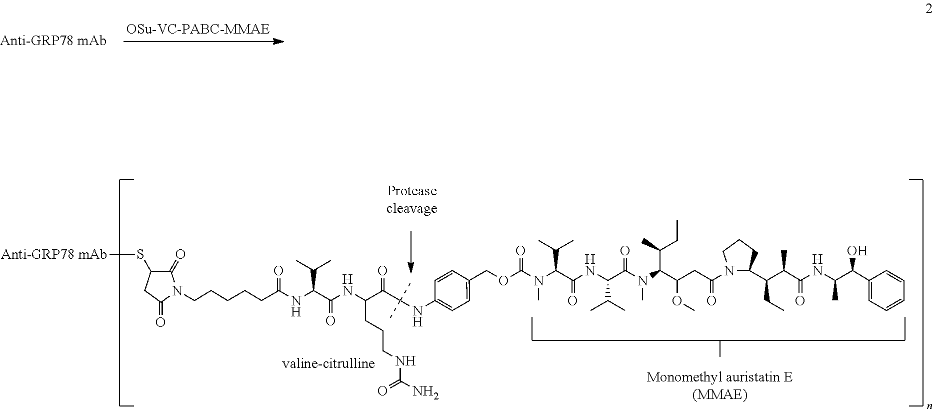

Linkers in ADCs may have significant impacts on the biological activities. For example, in vivo studies demonstrated that the peptide-linked conjugates induced regressions and cures of established tumor xenografts with therapeutic indices as high as 60-fold. These conjugates illustrate the importance of linker technology, drug potency and conjugation methodology in developing safe and efficacious mAb-drug conjugates for cancer therapy.

Some embodiments of the invention relate to MMAEs linked to antibodies via a lysosomally cleavable dipeptide, valine-citrulline (VC or Val-Cit), which have been shown to improve ADC efficacies. In this example, to a solution of Anti-GRP78 monoclonal antibody (400 μL (5.0 mg/mL) in buffer (50 mM potassium phosphate, 50 mM sodium chloride, 2 mM EDTA; pH 6.5) was slowly added 40 μL OSu-VC-PABC-MMAE (5 mM in DMSO). The reaction mixture was stirred under argon at 37° C. and stirred for 20 hours. The antibody preparation was desalted and concentrated using the AMICON™ Ultra-15 centrifugal filter device with 30 kDa NMWL in pH 7.4 PBS buffer to give Anti-GRP78 Antibody-VC-PABC-MMAE ADC 2.

### Example 6 Preparation of Anti-GRP78 Antibody-VC-PABC-Dxd Conjugates

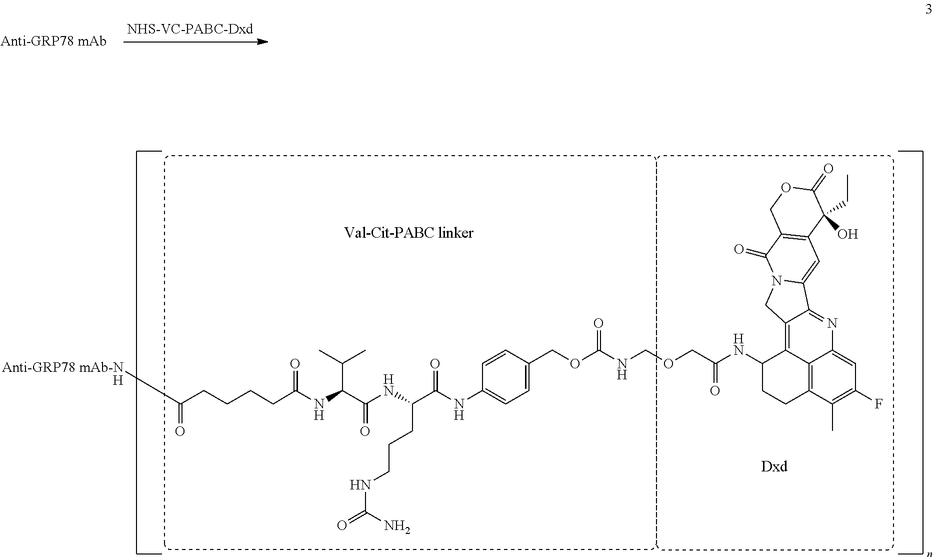

In this example, ADC contains Dxd, which is derivative of exatecan developed for cancer therapy. DXd is a potent DNA topoisomerase I inhibitor that interferes with DNA replication by stabilizing the DNA-topoisomerase I complex, leading to DNA damage and tumor cell death. This mechanism makes DXd highly effective in targeting rapidly dividing cancer cells.

DXd is able to be conjugated to antibodies via a cleavable linker to create antibody-drug conjugates (ADCs). These ADCs are designed to deliver Dxd selectively to tumor cells, minimizing systemic toxicity. For example, in trastuzumab deruxtecan, Dxd was linked with cleavable Glycine-Glycine-Phenylalanine-Glycine (GGFG) linker and conjugated to trastuzumab through Cys conjugation. The linker-payload of trastuzumab deruxtecan showed great plasma stability and enable the ADC to target the cancer cell to release the cytotoxic payload.

Some embodiments of the invention relate to Dxd linked to antibodies via succinimide ester (Lys conjugation) has demonstrated great cancer killing effect. In this example, to a solution of Anti-GRP78 monoclonal antibody (20 mL; 2.5 mg/mL) in buffer (25 mM sodium citrate, pH 6.5) was slowly mixed with 4 mL N-hydrozysuccinimide (NHS)-VC-para-aminobenzyl carbamate (PABC)-Dxd (1 mM in DMA). The reaction mixture was stirred at 37° C. for 90 minutes. The antibody preparation was desalted and concentrated using the AMICON™ Ultra-15 centrifugal filter device with 10 kDa MWCO in 10 mM histidine, 6% sucrose, pH 5.5 buffer to give Anti-GRP78-VC-PABC-Dxd ADC 3.

### Example 7 Binding Affinity of G1D10 and G1D10-ADC

### 7-1 the Recombinant GRP78 Protein Immobilization

This ligand has the optimum pH 4.0 condition for direct immobilization by a pH scouting test. With an amine coupling method, carboxyl groups on the surface of the sensor chip surface are first activated with a mixture of EDC and NHS to give reactive succinimide esters. GRP78 is then passed over the surface and the esters react with primary amine groups to link the ligand covalently to the dextran matrix. The target level of the GRP78 protein is about 300 RU.

### 7-2 the Binding Curve of G1D10 or G1D10-ADC to Recombinant GRP78

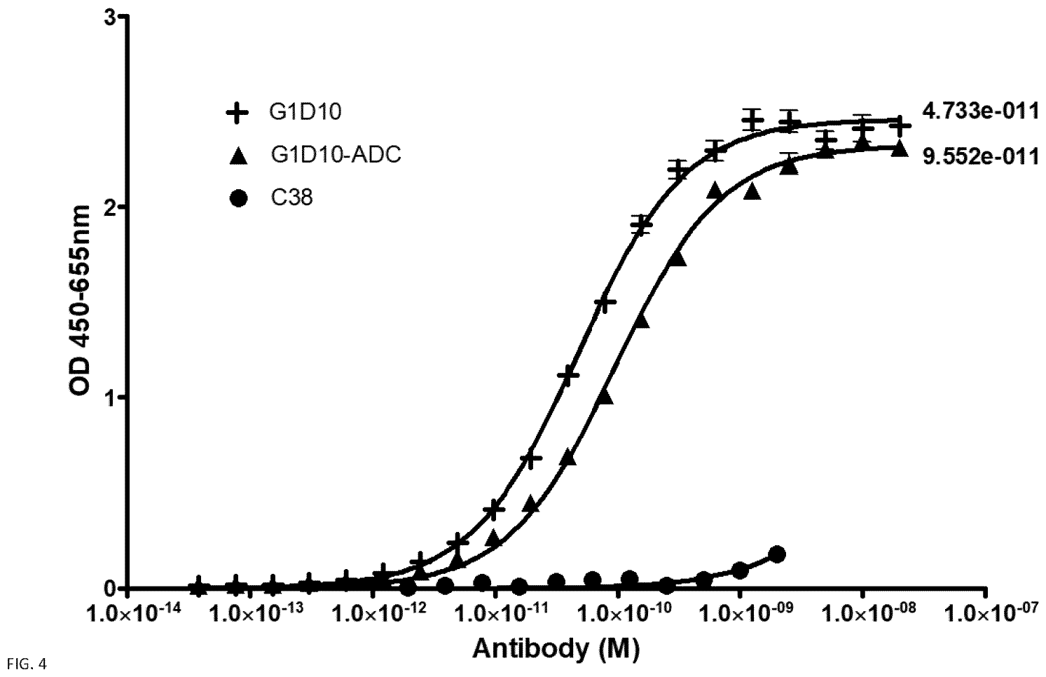

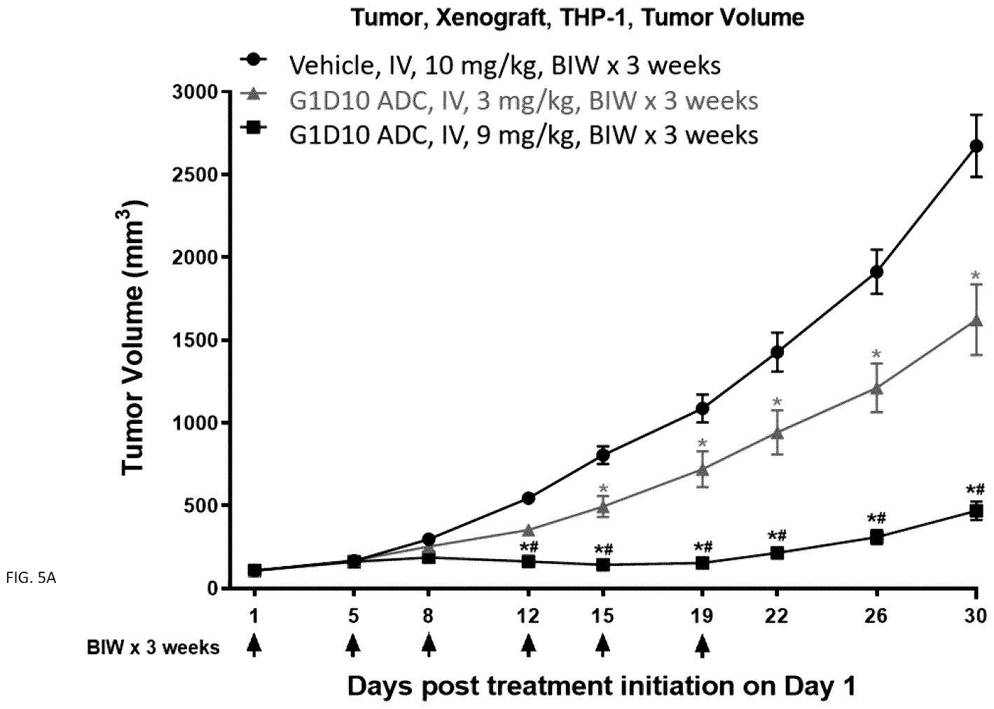

The antigen, GRP78, diluted in sodium carbonate buffer (pH 9.5), was coated on a 96-well plate at 4° C. overnight. After blocking (with 3% BSA), two-fold serial diluted GRP78 antibodies G1D10, GID10-ADC or C38 (EBIOSCIENCE™, Cat. No. 14-9768-82) were added to the wells and incubated at 37° C. for 1 hr. After binding, Goat anti-human IgG(H+L)-HRP (1:15000) (JACKSON IMMUNORESEARCH LAB™, Cat No. 109-035-003) and Goat anti-mouse IgG(H+L)-HRP (1:10000) (BETHYL™, Cat No. A90-216P) was added to GID10, GID10-ADC and C38 respectively and incubated at 37° C. for 1 hr. Then, 3,3′,5,5′-Tetramethylbenzidine (TMB) (SERA CARE™, Cat No. 5120-0077) substrate was used to develop colors and the reaction was stopped by addition of 1N HCl. The extents of antigen-antibody bindings were determined by measuring absorbance at 450-655 nm, using an ELISA reader (BIO-RAD™ iMark microplate absorbance reader). Data was analyzed using GraphPad Prism 5 software and shown in FIG. 4.

### Example 8 In-Vitro Cytotoxicity Assays of G1D10-ADC

In-Vitro cytotoxicity assays of GID10-ADC, DM1 and GID10 on HL-60, HCC1806, MDA-MB-231, Raji, RPMI8226, NCI-H929, MM.1S, G-361, THP-1, PL45, AsPC-1, KLM-1, OV90, 4Tl and EL-4 cells. Cell viability was measured after 48 hours using Cell Counting Kit-8 (CCK-8) assay (DOJINDO™, Cat. No. CK04-13), as shown in Table 3. For example, on Day 0, THP-1 cells were seeded at 5×10+ cells per well in a 96-well plate and treated with diluted test compounds. The cells were incubated for 48 hours at 37° C. with 5% CO₂. On Day 2, 20 μl of CCK-8 was added to each well, followed by incubation for 1-4 hours. Absorbance at 450 nm was then measured to assess cell viability and the impact of the test compounds. For suspension cells, such as HL-60, Raji, RPMI8226, NCI-H929, MM. 1S, and THP-1, 5×10⁴ cells per well were inoculated into a 96-well plate. For adherent cells, such as HCC1806, MDA-MB-231, G361, PL45, AsPC-1, KLM-1, OV90, and EL-4, 1.5-5×10³ cells per well were inoculated based on their doubling time. Adherent cells with a doubling time of less than 24 hours were seeded at 1.5×10³ cells per well (EL-4), while those with a doubling time of more than 24 hours were seeded at 5×10³ cells per well. In-vitro cytotoxicity assays showed that G1D10-ADC exhibited significantly greater cell-killing activity than the antibody alone. DM1 was used as the positive control agent. The cell lines tested included HL-60, HCC1806, MDA-MB-231, Raji, RPMI8226, NCI-H929, MM.1S, G-361, THP-1, PL45, AsPC-1, KLM-1, OV90, 4T1, and EL-4 cells. Cell viability was measured after 48 hours using the Cell Counting Kit-8 (CCK-8) assay (DOJINDO™, Cat. No. CK04-13). IC₅₀ (M) was calculated to determine the efficacy of the antibody, DM1, and ADC on the various cancer cell lines. The results across these cell lines demonstrated that G1D10-ADC has significant tumor cell-killing activity at the nanomolar level, indicating great potential in animal models and clinical applications.

TABLE 3

| | | IC₅₀ (M) | | |
|---|---|---|---|---|
| | | G1D10 | | G1D10 |
| Cancer Type | Cell Line | ADC (DM1) | DM1 | mAb |
| Lung Cancer | H460 | 5.969 × 10⁻⁸ | 1.681 × 10⁻⁹ | — |
| TNBC | HCC1806 | 6.413 × 10⁻⁸ | 2.722 × 10⁻⁸ | — |
| | MDA-MB- | 9.086 × 10⁻⁸ | 2.852 × 10⁻⁸ | — |
| | 231 | | | |
| DLBCL | Raji | 4.398 × 10⁻⁸ | 4.347 × 10⁻⁹ | — |
| MM | RPMI8226 | 5.056 × 10⁻⁹ | 1.793 × 10⁻⁹ | — |
| | NCI-H929 | 1.291 × 10⁻⁸ | 1.125 × 10⁻⁹ | — |
| | MM.1S | 2.551 × 10⁻⁸ | 4.859 × 10⁻⁹ | — |
| Melanoma | G361 | 6.306 × 10⁻⁸ | 3.106 × 10⁻⁹ | — |
| AML | THP-1 | 5.093 × 10⁻⁸ | 4.633 × 10⁻⁹ | — |
| Pancreatic | ASPC1 | 5.686 × 10⁻⁷ | 6.023 × 10⁻⁸ | — |
| Cancer | KLM-1 | 3.724 × 10⁻⁸ | 1.605 × 10⁻⁹ | — |
| Ovarian Cancer | OV90 | 1.944 × 10⁻⁷ | 5.521 × 10⁻⁸ | — |
| Murine Breast | 4T1 | 9.052 × 10⁻⁷ | 8.297 × 10⁻⁸ | — |
| Cancer | | | | |
| (Stage IV) | | | | |
| Murine | EL4 | 3.109 × 10⁻⁸ | 1.520 × 10⁻⁹ | — |
| Lymphoma | | | | |

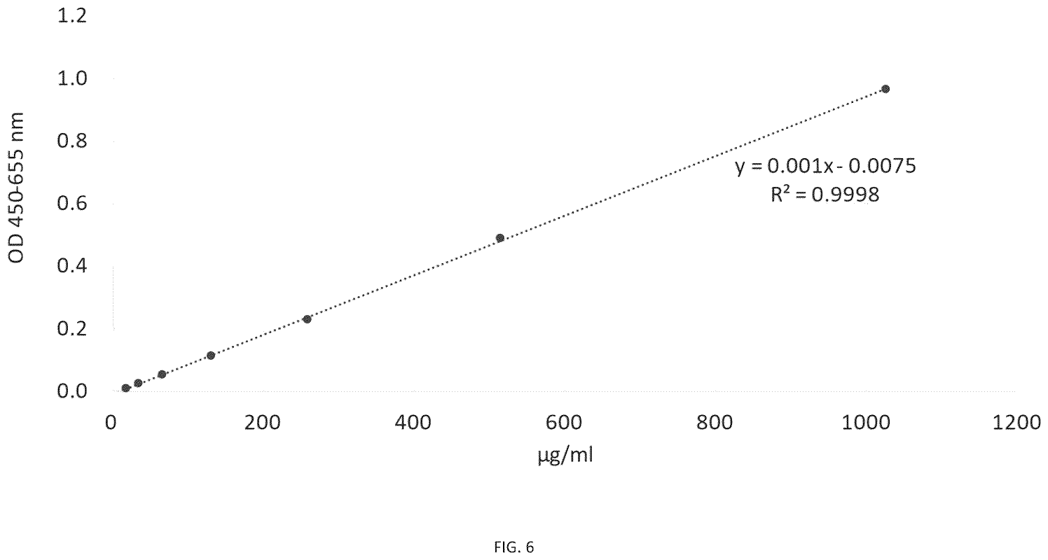

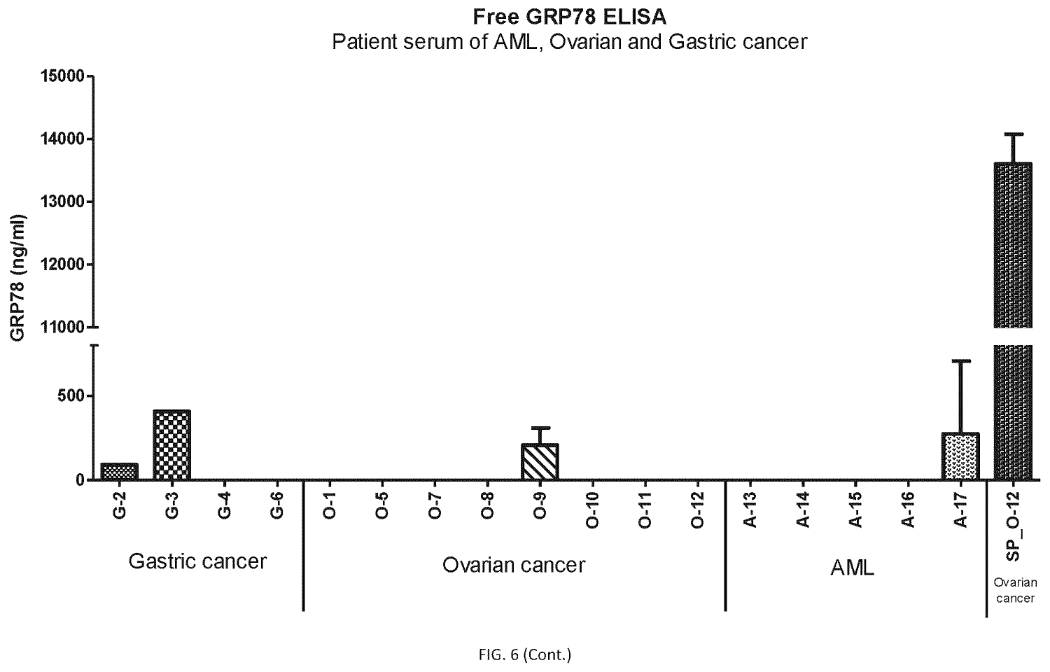

In-Vitro cytotoxicity assays of Dxd-conjugated ADC and free payload Dxd on THP-1, NCI-N87, Capan-1, and KLM-1 cells. Cell viability was measured after 48 hours using Cell Counting Kit-8 (CCK-8) assay (DOJINDO™, Cat. No. CK04-13), as shown in Table 4. For example, on Day 0, THP-1 cells were seeded at 5×10⁴ cells per well in a 96-well plate and treated with diluted test compounds. The cells were incubated for 48 hours at 37° C. with 5% CO₂. On Day 2, 15 μl of CCK-8 was added to each well, followed by incubation for 1-4 hours. Absorbance at 450 nm was then measured to assess cell viability and the impact of the test compounds. For suspension cells, such as THP-1, 2-5×10⁴ cells per well were inoculated into a 96-well plate. For adherent cells, such as NCI-N87, Capan-1, and KLM-1, 1.5-5×10³ cells per well were inoculated based on their doubling time. As shown in FIGS. 6A-6C, THP-1, and KLM-1 cells are not killed in the presence of G1D10 antibody alone. In-vitro cytotoxicity assays showed that G1D10-ADC (Dxd) exhibited cell-killing activity to those G1D10 antibody non-responsive cell lines. Linker-Dxd was used as the positive control agent. The cell lines tested included THP-1, NCI-N87, Capan-1, and KLM-1 cells. Cell viability was measured after 48 hours using the Cell Counting Kit-8 (CCK-8) assay (DOJINDO™, Cat. No. CK04-13). IC₅₀ (M) was calculated to determine the efficacy of the Linker-Dxd and the Dxd-conjugated ADC on the various cancer cell lines. The results across these cell lines demonstrated that G1D10-ADC (Dxd) has significant tumor cell-killing activity at the nanomolar level, indicating great potential in animal models and clinical applications.

TABLE 4

| | | IC₅₀ (M) | |
|---|---|---|---|
| Cancer Type | Cell Line | G1D10 ADC (Dxd) | Linker-Dxd |
| AML | THP-1 | 3.108 × 10⁻⁹ | 6.120 × 10⁻⁸ |
| Gastric Cancer | NCI-N87 | 4.971 × 10⁻⁸ | 1.614 × 10⁻⁷ |
| Pancreatic Cancer | Capan-1 | 8.237 × 10⁻⁸ | 2.956 × 10⁻⁷ |
| | KLM-1 | 6.725 × 10⁻⁸ | 1.842 × 10⁻⁷ |

### Example 9 In-Vivo Antitumor Activity of G1D10-ADC in THP-1 Xenograft Animal Model

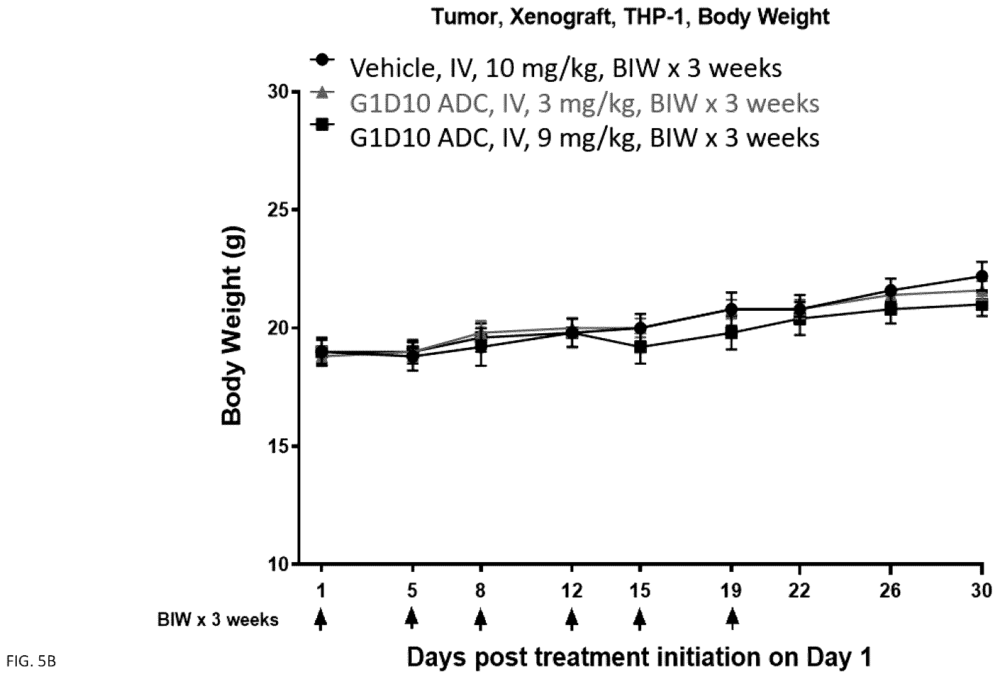

Anti-tumor effect of G1D10-ADC in THP-1 xenograft mouse model. Female SCID mice, 6-7 weeks of age, were subcutaneously implanted with viable human AMOL (AML/M5) THP-1 leukemia cells (ATCC TIB-202) at 1×10⁷ cells per mouse with 50% Matrigel in 0.2 mL volume, into the right flank. Once the average tumor volume reached 100 mm³, all animals were grouped into three groups (N=5), consisting of female SCID mice. THP-1 xenograft mice were administered via tail vein injection of G1D10-ADC at doses of 3 and 9 mg/kg (MPK), twice a week (BIW) for a total of six doses. The means±standard errors of tumor growth and body weight are shown in FIGS. 5A and 5B. [The length and width of tumors were measured with a caliper. The length represents the largest tumor diameter, and the width represents the perpendicular tumor diameter. The tumor volume was calculated according to the prolate ellipsoid formula: Tumor volume=length×(width) 2× 0.5.]

In the 3 mg/kg G1D10-ADC group, the mean tumor volume on Day 15 was significantly reduced compared to the vehicle control group (p<0.05). (*) indicates that the reduction was significant based on two-way ANOVA with Bonferroni correction. In the 9 mg/kg G1D10-ADC group, significant anti-tumor activity was observed on Day 12 compared to the vehicle control group. (#) indicates significant anti-tumor activity based on a T/C value≤42%. There are no significant changes in body weight throughout the study. This study demonstrates the anti-tumor efficacy of G1D10-ADC via IV injection (BIW for 3 weeks) and shows great potential for other animal models and clinical applications.

### Example 10 Patient GRP78 Detection with HRP-labeled G1D10 Immunoconjugate

For the diagnosis purpose, the anti-GRP78 mAb can be conjugated with various labels, such as radioisotope, fluorescence chemical, fluorescence protein, or HRP, to detect the expression of GRP78 on the cancer cells from patient specimens. However, for some cancer-specific/associated antigens, such as GRP78, they can be shed from the surface of cancer cell and enter the blood stream. The shed cancer-specific/associated antigen can bind to the therapeutic antibody and serve as decoy receptor to block the function of the therapeutics. Therefore, to determine the level of shed cancer-specific/associated antigen in patient's blood sample can be used to ensure that the minimum effective dose of the therapeutics can be reached after interacting with the shed cancer-specific/associated antigen.

To determine the level of GRP78 in the patient's blood sample, the anti-GRP78 mAb G1D10 was labeled with HRP by using the commercial kit (EZ-Link™ Plus Activated Peroxidase Kit, Thermo Scientific). 2 μg/mL of the anti-GRP78 mAb GD17 (homemade, refer to EP2130552B1) was coated in the well of ELISA plate at 4° C. overnight. The plate was washed and blocked with blocking buffer (5% skim milk in PBS) at 37° C. for 1 hour. Free GRP78 with various concentration or diluted patient serum was applied and incubated with the coated antibody at 37° C. for 2 hours. The captured GRP78 was detected by the HRP-labeled anti-GRP78 mAb G1D10 (G1D10-HRP) at 37° C. for 1 hour followed by adding TMB and in turn 1N HCl. By following the ELISA protocol, the calibration standard curve can be established, and the free GRP78 in the serum from various cancer patients, including AML, ovarian cancer, and gastric cancer, can be analyzed. The GRP78 protein was added into the patient serum (SP_O-12) and used as the positive control as shown in FIG. 6.

1. An isolated antibody or antigen-binding fragment thereof that is specific to an epitope in glucose-related protein 78 (GRP78); wherein the isolated antibody or antigen-binding fragment thereof comprises complementarity determining regions (CDRs) of a heavy chain variable region and/or CDRs of a light chain variable region,
wherein the CDRs of the heavy chain variable region comprise:
CDRH1 having the amino acid sequence of SEQ ID NO: 1 or the amino acid sequence with at least about 95% identity to SEQ ID NO: 1, CDRH2 having the amino acid sequence of SEQ ID NO: 2 or the amino acid sequence with at least about 95% identity to SEQ ID NO: 2, and CDRH3 having the amino acid sequence of SEQ ID NO: 3 or the amino acid sequence with at least about 95% identity to SEQ ID NO: 3; and
wherein the CDRs of the light chain variable region comprise:
CDRL1 having the amino acid sequence of SEQ ID NO: 4 or the amino acid sequence with at least about 95% identity to SEQ ID NO: 4, CDRL2 having the amino acid sequence of SEQ ID NO: 5 or the amino acid sequence with at least about 95% identity to SEQ ID NO: 5, and CDRL3 having the amino acid sequence of SEQ ID NO: 6 or the amino acid sequence with at least about 95% identity to SEQ ID NO: 6.

2. The isolated antibody or antigen-binding fragment thereof of claim 1, wherein the heavy chain variable region comprises the amino acid sequence of SEQ ID NO: 7 or the amino acid sequence with at least about 95% identity to SEQ ID NO: 7; or the light chain variable region comprises the amino acid sequence of SEQ ID NO: 8 or the amino acid sequence with at least about 95% identity to SEQ ID NO: 8.

3. The isolated antibody or antigen-binding fragment thereof of claim 1, which is an Fab fragment, an F(ab′) 2 fragment, an scFv fragment, a monoclonal antibody, a nanobody, a chimeric antibody, a composite antibody, a humanized antibody or a human antibody.

4. The isolated antibody or antigen-binding fragment thereof of claim 1, which is expressed on a surface of a cell.

5. The isolated antibody or antigen-binding fragment thereof of claim 4, wherein the cell is an immune cell or a stem cell.

6. A genetically engineered cell expressing the isolated antibody or antigen-binding fragment thereof of claim 1.

7. The genetically engineered cell of claim 6, which is an immune cell or a stem cell.

8. An immunoconjugate that binds specifically to GRP78, comprising:
the isolated antibody or antigen-binding fragment thereof of claim 1, and
a therapeutic agent or a label,
wherein the immunoconjugate has the formula of
Ab-(L-D)ₘ,
wherein Ab represents the isolated antibody or antigen-binding fragment thereof;
L represents a linker or a direct bond;
D represents the therapeutic agent or the label; and
m represents an integer from 1 to 15.

9. The immunoconjugate thereof claim 8, wherein the linker is a hydrazone linker, a disulfide bond linker, a maleimidomethyl cyclohexane-1-carboxylate (MCC) linker, a maleimidocaproyl (MC) linker, a Val-Cit (valine-citrulline, VC) linker, a VC-PABC (valine-citrulline-p-aminobenzyl carbamate) linker, a GGFG (Gly-Gly-Phe-Gly) linker, an acid liable carbonate linker, or a sulfo-SPDB (N-succinimidyl 4-(2-pyridyldithio)-2-sulfobutanoate) linker.

10. The immunoconjugate thereof claim 8, wherein the label is a radioisotope, a fluorescence, or a reporter enzyme.

11. The immunoconjugate thereof claim 8, wherein the therapeutic agent is a calicheamicin, MMAE (monomethyl auristatin E), MMAF (monomethyl auristatin F), emtansine (DM1), ravtansine (DM4), pyrrolobenzodiazepine (PBD) dimer, exatecan, 7-ethyl-10-hydroxycamptothecin (SN38), or deruxtecan (Dxd).

12. The immunoconjugate thereof claim 8, wherein the therapeutic agent is Dxd and the linker is VC, VC-PABC, or GGFG.

13. The immunoconjugate thereof claim 8, wherein the linker is conjugated to the isolated antibody or antigen-binding fragment thereof through lysine-conjugation or cysteine-conjugation.

14. A method for use in treating, prophylactically treating and/or preventing a disease and/or disorder caused by or related to GRP78 activity or signaling in a subject in need thereof, comprising administering the subject an effective amount of the isolated antibody or antigen-binding fragment thereof of claim 1 and optionally a pharmaceutically acceptable carrier.

15. The method of claim 14, wherein the disease is a cancer.

16. A method for treating, prophylactically treating and/or preventing a disease and/or disorder caused by or related to GRP78 activity or signaling in a subject in need thereof, comprising administering the subject an effective amount of the genetically engineered cell of claim 6 and optionally a pharmaceutically acceptable carrier.

17. The method of claim 16, wherein the disease is cancer.

18. A method for treating, prophylactically treating and/or preventing a disease and/or disorder caused by or related to GRP78 activity or signaling in a subject in need thereof, comprising administering the subject an effective amount of the immunoconjugate of claim 8 and optionally a pharmaceutically acceptable carrier.

19. The method of claim 18, wherein the disease is cancer.

20. A method for detecting GRP78 or cancer in a sample, comprising contacting the sample with the isolated antibody or antigen-binding fragment thereof of claim 1.


SEQUENCE LISTING (12 sequences)

SEQ ID NO: 1 - protein, 10 aa, Homo sapiens
GFTFSNYAMN

SEQ ID NO: 2 - protein, 17 aa, Homo sapiens
IIYPDSSATYYADSVKG

SEQ ID NO: 3 - protein, 10 aa, Homo sapiens
YHFRWGAMDY

SEQ ID NO: 4 - protein, 11 aa, Homo sapiens
RASQGGSRNLN

SEQ ID NO: 5 - protein, 7 aa, Homo sapiens
KNSNLQS

SEQ ID NO: 6 - protein, 9 aa, Homo sapiens
QQYGSLPWT

SEQ ID NO: 7 - protein, 119 aa, Homo sapiens
EVQLVESGGGLVQPGGSLRLSCAASGFTFSNYAMNWVRQAPGKGLEWVSIIYPDSSATYYADSVKGRFTISRDNSKNTLYLQMNSLRAEDTAVYYCARYHFRWGAMDYWGQGTLVTVSS

SEQ ID NO: 8 - protein, 108 aa, Homo sapiens
DIQMTQSPSSLSASVGDRVTITCRASQGGSRNLNWYQQKPGKAPKLLIYKNSNLQSGVPSRFSGSGSGTDFTLTISSLQPEDFATYYCQQYGSLPWTFGQGTKVEIKR

SEQ ID NO: 9 - protein, 448 aa, Homo sapiens
EVQLVESGGGLVQPGGSLRLSCAASGFTFSNYAMNWVRQAPGKGLEWVSIIYPDSSATYYADSVKGRFTISRDNSKNTLYLQMNSLRAEDTAVYYCARYHFRWGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Homo sapiens
DIQMTQSPSSLSASVGDRVTITCRASQGGSRNLNWYQQKPGKAPKLLIYKNSNLQSGVPSRFSGSGSGTDFTLTISSLQPEDFATYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1347 bp, synthetic construct
  source 1..1347: mol_type = other DNA
gaagttcagctggttgaaagcggcggtggtctggttcagcctggtggcagcctgcgcctgagctgtgcagcaagcggctttaccttttctaactacgcaatgaactgggttcgccaggcacctggcaaaggcctggaatgggttagcatcatttacccagattcttctgcaacctactatgcagatagcgttaaaggccgctttaccattagccgcgataatagcaaaaataccctgtacctgcaaatgaacagcctgcgcgcagaggataccgccgtgtactattgtgcccgctaccacttccggtggggggctatggattattggggccagggcaccctggtcaccgtgagctctgctagcaccaagggcccttccgtgttccctctggccccttccagcaagtctacctctggcggcaccgctgctctgggctgcctcgtgaaggactacttccctgagcctgtgacagtgtcctggaactctggcgccctgacctccggcgtgcacaccttccctgccgtgctgcagtcctccggcctgtactctctgtcctccgtcgtgacagtgccttcctccagcctgggcacccagacctacatctgcaacgtgaaccacaagccttccaacaccaaggtggacaagaaggtggagcctaagtcctgcgacaagacccacacctgtcctccatgccctgcccctgagctgctgggcggaccctccgtgttcctgttccctccaaagcctaaggacaccctgatgatctcccggacccctgaagtgacctgcgtggtggtggacgtgtcccacgaggaccctgaagtgaagttcaattggtacgtggacggcgtggaggtgcacaatgctaagaccaagcctcgggaggaacagtacaactccacctaccgggtggtgtccgtgctgaccgtgctgcaccaggactggctgaacggcaaagaatacaagtgcaaggtgtccaacaaggccctgcctgcccctatcgaaaagaccatctccaaggccaagggccagcctcgggaacctcaggtgtacaccctgcctcccagcagggaggagatgaccaagaaccaggtgtccctgacctgtctggttaagggcttctacccttccgacatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtctctgtctcctggctga

SEQ ID NO: 12 - DNA, 645 bp, synthetic construct
  source 1..645: mol_type = other DNA
gatatccagatgacacagagccctagcagcctgtctgccagcgtgggcgatcgcgtgaccatcacctgtagagccagccagggtggttctcgcaacctgaactggtatcagcagaagcctggcaaagcccctaaactgctgatctataaaaactctaacctgcagtctggcgtgccaagccgcttttctggcagcggctctggcaccgacttcaccctgaccatctctagcctgcagcctgaagacttcgccacctattattgccagcagtacggttctttgccatggacctttggccagggcaccaaagtggaaatcaaacgcacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga